(19) United States
(12) Patent Application Publication
(10) Pub. No.: US 2020/0138457 A1
(43) Pub. Date: May 7, 2020

(54) SURGICAL DRILL GUIDE TOOLS

(71) Applicant: LOREAN DYNAMIC SURGICAL NAVIGATION LTD., Tiberias (IL)

(72) Inventors: Adi LOREAN, Tiberias (IL); Noa BARER, Hod Hasharon (IL); Ori DAR, Kefar Saba (IL)

(73) Assignee: LOREAN DYNAMIC SURGICAL NAVIGATION LTD., Tiberias (IL)

(21) Appl. No.: 16/607,950

(22) PCT Filed: Apr. 26, 2018
(86) PCT No.: PCT/IL2018/050458

§ 371 Date: Oct. 24, 2019

Related U.S. Application Data
(60) Provisional application No. 62/490,118, filed on Apr. 26, 2017.

(51) Int. Cl. A61B 17/17; A61B 17/16; A61B 17/32; A61B 18/20; A61C 1/08; A61C 8/00

(52) U.S. Cl. CPC A61B 17/17; A61B 17/1615; A61B 17/320068; A61B 18/20; A61B 2018/00601; A61C 8/0034; A61B 2017/1602; A61B 2017/320052; A61C 1/084

(57) ABSTRACT

A surgical cutting tool guide (20, 320, 420, 520) includes a target arm (30) and primary and secondary sight arms (32A, 32B), which are pivotably attached to the target arm (30) so as to be rotatable with respect to one another about primary and secondary target pivot points (34A, 34B), respectively. A sight (40) includes a sight housing (42), which defines a sight passage (44) for aligning a surgical cutting tool (22) with a sight target axis (46), and is attached to the primary and the secondary sight arms (32A, 32B) such that the sight housing (42) and the primary and the secondary sight arms (32A, 32B), respectively, are rotatable with respect to each other about the primary and secondary sight pivot points (34A, 34B), respectively. The surgical cutting tool guide (20, 320, 420, 520) is arranged to automatically and non-electrically maintain the sight target axis (46) oriented to pass within an offset distance of a target point (60) over an entire range of angles over which the sight (40) directs the sight passage (44) toward the target point (60).

### CROSS-REFERENCE TO RELATED APPLICATIONS

The present application claims priority from U.S. Provisional Application 62/490,118, filed Apr. 26, 2017, which is assigned to the assignee of the present application and is incorporated herein by reference.

### FIELD OF THE APPLICATION

The present invention relates generally to medical tools, and specifically to medical drill guide tools for guiding cutting tools during surgical procedures.

### BACKGROUND OF THE APPLICATION

Zygomatic dental implants are anchored in the zygomatic bone (cheek bone) rather than the maxilla. They may be used when maxillary bone quality or quantity is inadequate for the placement of conventional dental implants. One complication of zygomatic implants is orbital cavity penetration, which may occur if the drilling direction is not precisely controlled.

### SUMMARY OF THE APPLICATION

Embodiments of the present invention provide surgical cutting tool guides, for use with a surgical cutting tool, such as a drill bit. For some applications, the surgical cutting tool guides are used to drill a bore into a zygomatic bone for anchoring a zygomatic implant.

The surgical cutting tool guide typically comprises:
a target arm;
one or more sight arms; and
a sight, which is attached to the one or more sight arms, and which comprises a sight housing, which typically defines a sight passage for aligning the surgical cutting tool with a sight target axis.

The surgical cutting tool guide is arranged to permit relative movement between the sight and the target arm. Typically, the surgical cutting tool guide further comprises a target-stabilization unit, which is disposed at a distal end of the target arm, and is configured to stabilize the target point with respect to a body of a patient, such as with respect to the zygomatic bone. Typically, the surgical cutting tool guide is arranged to automatically and non-electrically maintain the sight target axis oriented to pass through the target-stabilization unit.

For some applications, the target-stabilization unit is shaped so as to define a target housing that is disposed around at least 180 degrees (e.g., at least 270 degrees, such as 360 degrees) of the target point (and the sight target axis). The target housing visually indicates the location of the target point to the surgeon. For some applications, the target housing is shaped so as to define a surface facing the target point (and the sight target axis). Typically, the target housing entirely surrounds the target point (and the sight target axis), such as shown in the figures. Typically, the target point (and the sight target axis) is centered in the target housing. For some applications, the target housing is shaped as a ring, and, optionally, the target point (and the sight target axis) is centered in the ring. Optionally, a surface of the target housing that faces the sight housing, and thus is pressed against tissue of the subject, is soft or padded.

For some applications, the one or more sight arms comprise:
a primary sight arm, which is pivotably attached to the target arm such that the primary sight arm and the target arm are rotatable with respect to each other about a primary target pivot point, so as to permit relative movement between the primary sight arm and the target arm; and
a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm.

The sight housing is attached to:
the primary sight arm such that the sight housing and the primary sight arm are rotatable with respect to each other about a primary sight pivot point, and
the secondary sight arm such that the sight housing and the secondary sight arm are rotatable with respect to each other about a secondary sight pivot point that does not coincide with the primary sight pivot point.

The surgical cutting tool guide is arranged to automatically and non-electrically maintain the sight target axis oriented to pass within an offset distance of a target point over an entire range of angles that extends at least between 11 degrees and 22 degrees. The surgical cutting tool guide maintains this orientation by constant relative motion between certain elements of the surgical cutting tool as the arms are moved with respect to one another. The angles correspond to respective different orientations of the primary sight arm with respect to the target arm (and to respective different orientations of the secondary sight arm with respect to the target arm). The angles are measured between (a) a first line that is defined by the target point and the primary target pivot point and (b) a second line that (i) intersects the first line and (ii) intersects and is perpendicular to the sight target axis. Typically, an intersection point at which the first and the second lines intersect moves along the first line and/or the second line over the entire range of angles.

Typically, the offset distance equals less than 5%, such as less than 3%, e.g., less than 1% (such as less than 0.75%, e.g., less than 0.5%, ideally 0%) of a target axis distance measured between the target point and the primary target pivot point, such that the sight directs the sight passage (and the sight target axis) toward the target point (rather than toward an axis) over the entire range of angles. As a result, the sight passage guides the surgical cutting tool toward the target point (within the offset distance) regardless of which angle is set from the entire range of angles α (alpha).

Typically, the primary target pivot point, the secondary target pivot point, the primary sight pivot point, and the secondary sight pivot point (a) are not arranged as a parallelogram at any of the angles of the above-mentioned entire range of angles between the first and the second lines, and (b) are arranged a quadrilateral.

For some applications, the secondary sight arm is farther from the first line than the primary sight arm is from the first line. In other words, the primary sight arm is generally between the secondary sight arm and the first line. For other applications, the secondary sight arm is closer to the first line than the primary sight arm is to the first line.

For some applications, the surgical cutting tool guide comprises a plurality of visually-sensible fiducial designators disposed about the primary target pivot point, which indicate a measurement angle between (a) the first line and (b) a fourth line defined by the primary sight pivot point and the primary target pivot point.

For some applications, the surgical cutting tool guide comprises:
a first joint that facilitates relative rotation of the target housing about the first line over a first entire range of angles that extends at least 90 degrees (e.g., at least 120 degrees); the target point moves, with respect to the target housing, by less than 2% of the target axis distance during rotation of the target housing over the first entire range of angles;
a second joint that facilitates relative rotation of the target housing about a line perpendicular to both the first line and the sight target axis over a second entire range of angles that extends at least 90 degrees (e.g., at least 120 degrees); the target point moves, with respect to the target housing, by less than 2% of the target axis distance during rotation of the target housing over the second entire range of angles; and/or
a third joint that facilitates relative rotation of an element of the target housing about the sight target axis over a third entire range of angles that extends at least 90 degrees (e.g., at least 120 degrees, such as 360 degrees); the target point moves, with respect to the target housing, by less than 2% of the target axis distance during rotation of the target housing over the third entire range of angles.

For other applications, the surgical cutting tool guide further comprises a main body, which comprises an elongate support structure. A proximal portion of the target arm is attached to the main body. The one or more sight arms are slidably attached to the elongate support structure, such that the one or more sight arms can move linearly and assume a plurality of positions along the elongate support structure, thereby permitting the relative movement between the sight and the target arm. The surgical cutting tool guide is arranged to automatically and non-electrically maintain the sight target axis oriented to pass within an offset distance of the target point at all of the positions of the one or more sight arms along the elongate support structure. Typically, the offset distance equals less than 2.5 mm, such as less than 1.5 mm. For some applications, the main body comprises a mechanical control element that is disposed remotely from the one or more sight arms, and is configured to mechanically and non-electrically linearly move the one or more sight arms along the elongate support structure.

For some applications, the surgical cutting tool guide is used for drilling a bore into a zygomatic bone for anchoring a zygomatic implant. The sight, as well as portions of the one or more sight arms, are inserted through the mouth into an oral cavity. The target point is positioned at or near a malar point (the most prominent point on the zygomatic bone). For some applications, the target-stabilization unit is placed against skin of a cheek, which both positions the target point and stabilizes the target point with respect to the zygomatic bone. The target-stabilization unit typically assumes an appropriate orientation by itself, because of the anatomy and the degrees of freedom of motion provided by the first joint, the second joint, and/or the third joint. After the target-stabilization unit has assumed the appropriate orientation, if provided, a first lock and/or a second lock are typically locked to maintain the orientation. Typically, the target housing is disposed against the skin partially or entirely around the malar point.

The sight is positioned near a desired implantation site of the maxilla (a working surface of the bone), and is pushed tightly against the implantation site by bringing the sight closer to the target-stabilization unit, by bringing the one or more sight arms closer to the target arm. For some applications, the one or more sight arms are brought closer to the target arm by bringing finger loops, which are disposed outside the oral cavity, closer together. The target-stabilization unit and the sight are thus arranged to grasp a bony part of the body therebetween. In configurations in which the surgical cutting tool guide comprises a locking mechanism, the locking mechanism locks the sight with respect to the target-stabilization unit, thereby maintaining the sight clamped tightly against the implantation site and the target-stabilization unit tightly against the skin of the cheek at or near the malar point, and the sight at a fixed position and orientation with respect to the anatomy, including the zygomatic bone.

As the sight is brought closer to the target-stabilization unit, the surgical cutting tool guide automatically and non-electrically maintains the orientation of the sight passage toward the target point, even as the angle changes as the arms are brought closer together.

As a result of these features, the surgeon can use a single hand for positioning and clamping the surgical cutting tool guide in the proper position for cutting into the zygomatic bone. The target point is ex vivo, and the surgical cutting tool guide provides non-invasive fixation of the target point. In addition, the surgeon does not need to calculate or otherwise ascertain the angle before the sight is inserted into the oral cavity.

The surgical cutting tool, such as a drill bit, is advanced through the sight passage. The sight passage directs the surgical cutting tool toward the target point (within the offset distance), regardless of the particular angle. The surgical cutting tool guide thus prevents accidental penetration of an orbital cavity by the surgical cutting tool. To the extent that any deviation may inadvertently occur using the surgical cutting tool guide, the deviation is lateral, rather than medial, anterior, or superior. The surgical cutting tool guide may also facilitate the performance of a flapless technique (in conventional zygomatic dental implant procedures, a flap is opened to visually check the trajectory of the drill).

There is therefore provided, in accordance with an Inventive concept 1 of the present invention, apparatus including a surgical cutting tool guide for use with a surgical cutting tool, the surgical cutting tool guide including:
a target arm;
a primary sight arm, which is pivotably attached to the target arm such that the primary sight arm and the target arm are rotatable with respect to each other about a primary target pivot point, so as to permit relative movement between the primary sight arm and the target arm;
a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm; and
a sight, which includes a sight housing, which (a) defines a sight passage for aligning the surgical cutting tool with a sight target axis, and (b) is attached to:
   the primary sight arm such that the sight housing and the primary sight arm are rotatable with respect to each other about a primary sight pivot point, and
   the secondary sight arm such that the sight housing and the secondary sight arm are rotatable with respect to each other about a secondary sight pivot point that does not coincide with the primary sight pivot point,
wherein the surgical cutting tool guide is arranged to automatically and non-electrically maintain the sight target axis oriented to pass within an offset distance of a target point over an entire range of angles that extends at least between 11 degrees and 22 degrees, wherein the angles correspond to respective different orientations of the primary sight arm with respect to the target arm and are measured between (a) a first line that is defined by the target point and the primary target pivot point and (b) a second line that (i) intersects the first line and (ii) intersects and is perpendicular to the sight target axis, and
wherein the offset distance equals less than 5% of a target axis distance measured between the target point and the primary target pivot point, such that the sight directs the sight passage toward the target point over the entire range of angles.
Inventive concept 2. The apparatus according to Inventive concept 1, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.
Inventive concept 3. The apparatus according to Inventive concept 1, wherein the offset distance equals less than 3% of the target axis distance.
Inventive concept 4. The apparatus according to Inventive concept 3, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.
Inventive concept 5. The apparatus according to Inventive concept 3, wherein the offset distance equals less than 1% of the target axis distance.
Inventive concept 6. The apparatus according to Inventive concept 5, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.
Inventive concept 7. The apparatus according to Inventive concept 5, wherein the offset distance equals less than 0.75% of the target axis distance.
Inventive concept 8. The apparatus according to Inventive concept 7, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.
Inventive concept 9. The apparatus according to Inventive concept 1, wherein the cutting tool is a drill bit, wherein the surgical cutting tool guide is a surgical drill guide, and wherein the sight passage is arranged to guide the surgical drill bit along the sight target axis.
Inventive concept 10. The apparatus according to Inventive concept 1, wherein the cutting tool is a milling bit, wherein the surgical cutting tool guide is a surgical milling guide, and wherein the sight passage is arranged to guide the surgical milling bit along the sight target axis.
Inventive concept 11. The apparatus according to Inventive concept 1, wherein the cutting tool is a laser cutting tool, wherein the surgical cutting tool guide is a surgical laser cutting tool guide, and wherein the sight passage is arranged to align the laser cutting tool along the sight target axis.
Inventive concept 12. The apparatus according to Inventive concept 1, wherein the cutting tool is an ultrasonic cutting tool, wherein the surgical cutting tool guide is a surgical ultrasonic cutting tool guide, and wherein the sight passage is arranged to align the ultrasonic cutting tool with the sight target axis.
Inventive concept 13. The apparatus according to Inventive concept 1, wherein a third distance between the primary sight pivot point and the primary target pivot point is not equal to a fourth distance between the secondary sight pivot point and the secondary target pivot point.
Inventive concept 14. The apparatus according to Inventive concept 13, wherein (a) the lesser of the third and the fourth distances is less than 98% of (b) the greater of the third and the fourth distances.
Inventive concept 15. The apparatus according to Inventive concept 13, wherein the third distance is less than the fourth distance.
Inventive concept 16. The apparatus according to Inventive concept 1, wherein the secondary sight arm is farther from the first line than the primary sight arm is from the first line.
Inventive concept 17. The apparatus according to Inventive concept 1, wherein the secondary sight arm runs at least partially alongside the primary sight arm.
Inventive concept 18. The apparatus according to Inventive concept 1, wherein the target arm and the primary sight arm are shaped so as to define respective finger loops.
Inventive concept 19. The apparatus according to Inventive concept 1, wherein the surgical cutting tool guide includes a plurality of visually-sensible fiducial designators disposed about the primary target pivot point, which indicate a measurement angle between (a) the first line and (b) a fourth line defined by the primary sight pivot point and the primary target pivot point.
Inventive concept 20. The apparatus according to Inventive concept 19, wherein the visually-sensible fiducial designators are arranged to display the measurement angle in a range of at least 25 degrees to 45 degrees.
Inventive concept 21. The apparatus according to any one of Inventive concepts 1-20, wherein the primary target pivot point, the secondary target pivot point, the primary sight pivot point, and the secondary sight pivot point (a) are not arranged as a parallelogram at any of the angles of the entire range of angles between the first and the second lines, and (b) are arranged a quadrilateral.
Inventive concept 22. The apparatus according to Inventive concept 21,
   wherein the quadrilateral has a first angle at the primary sight pivot point and a second angle at the secondary target pivot point, and
   wherein a difference between the first and the second angles equals at least 75 degrees over the entire range of angles between the first and the second lines.
Inventive concept 23. The apparatus according to any one of Inventive concepts 1-20, wherein the target arm includes a central housing, which includes the primary target pivot point and the secondary target pivot point.
Inventive concept 24. The apparatus according to Inventive concept 23,
   wherein the central housing includes an arm-adjustment mechanism, which includes a lead screw, which (a) is coupled to the central housing such that the lead screw can rotate with respect to the central housing, and (b) is shaped so as to define:
      a user-control knob,
      a threaded lead screw portion, wherein the central housing is shaped so as to define an internally threaded channel through which the threaded lead screw portion is disposed,
      first and second limiting radial protrusions at different first and second axial regions, respectively, along the lead screw,
   wherein a proximal end of the primary sight arm is shaped so as to define an engaging protrusion, which is aligned with an axial region of the lead screw that is axially between the first and the second limiting radial protrusions, and
   wherein the lead screw and the primary sight arm are arranged such that axial motion of the lead screw causes axial motion of the engaging protrusion, which in turn causes rotation of the primary sight arm about the primary target pivot point, wherein the rotation moves the primary sight arm with respect to the target arm.
Inventive concept 25. The apparatus according to any one of Inventive concepts 1-20, wherein the surgical cutting tool guide further includes a target-stabilization unit, which is disposed at a distal end of the target arm, and is configured to stabilize the target point with respect to a body of a patient.
Inventive concept 26. The apparatus according to Inventive concept 25, wherein the surgical cutting tool guide is arranged to automatically and non-electrically maintain the sight target axis oriented to pass through the target-stabilization unit over the entire range of angles.
Inventive concept 27. The apparatus according to Inventive concept 25, wherein the target-stabilization unit is shaped so as to define a target housing that is disposed around at least 180 degrees of the target point.
Inventive concept 28. The apparatus according to Inventive concept 27, wherein the target housing is shaped so as to define a surface facing the target point.
Inventive concept 29. The apparatus according to Inventive concept 27, wherein the target housing is disposed around at least 270 degrees of the target point.
Inventive concept 30. The apparatus according to Inventive concept 29, wherein the target housing entirely surrounds the target point.
Inventive concept 31. The apparatus according to Inventive concept 30, wherein the target housing is shaped so as to define an annular surface facing the target point.
Inventive concept 32. The apparatus according to Inventive concept 31, wherein the annular surface is circular or polygonal.
Inventive concept 33. The apparatus according to Inventive concept 30, wherein the target point is centered in the target housing.
Inventive concept 34. The apparatus according to Inventive concept 27, wherein the surgical cutting tool guide includes a joint that facilitates relative rotation of the target housing about the first line over an entire range of angles that extends at least 90 degrees, and wherein the target point moves, with respect to the target housing, by less than 2% of the target axis distance during rotation of the target housing over the entire range of angles.
Inventive concept 35. The apparatus according to Inventive concept 27, wherein the surgical cutting tool guide includes a joint that facilitates relative rotation of the target housing about a line perpendicular to both the first line and the sight target axis over an entire range of angles that extends at least 90 degrees, and wherein the target point moves, with respect to the target housing, by less than 2% of the target axis distance during rotation of the target housing over the entire range of angles.
Inventive concept 36. The apparatus according to Inventive concept 27, wherein the surgical cutting tool guide includes a joint that facilitates relative rotation of an element of the target housing about the sight target axis over an entire range of angles that extends at least 90 degrees, and wherein the target point moves, with respect to the target housing, by less than 2% of the target axis distance during rotation of the target housing over the entire range of angles.
Inventive concept 37. The apparatus according to Inventive concept 27,
   wherein the surgical cutting tool guide includes joints that facilitate relative rotation of the target housing about:
      the first line over a first entire range of angles that extends at least 90 degrees, and
      a line perpendicular to both the first line and the sight target axis over a second entire range of angles that extends at least 90 degrees, and
   wherein the target point moves, with respect to the target housing, by less than 2% of the target axis distance during rotation of the target housing over the first entire range of angles and the second entire range of angles.
Inventive concept 38. The apparatus according to Inventive concept 27,
   wherein the surgical cutting tool guide includes joints that facilitate relative rotation of:
      the target housing about the first line over a first entire range of angles that extends at least 90 degrees,
      the target housing about a line perpendicular to both the first line and the sight target axis over a second entire range of angles that extends at least 90 degrees,
      an element of the target housing about the sight target axis over a third entire range of angles that extends at least 90 degrees, and
   wherein the target point moves, with respect to the target housing, by less than 2% of the target axis distance during rotation of the target housing over the first entire range of angles, the second entire range of angles, and the third entire range of angles.
Inventive concept 39. The apparatus according to Inventive concept 25, wherein the target-stabilization unit and the sight are arranged to grasp a bony part of the body therebetween.
Inventive concept 40. The apparatus according to Inventive concept 39, wherein the surgical cutting tool guide further includes a locking mechanism, which is configured to lock the sight with respect to the target-stabilization unit.
Inventive concept 41. The apparatus according to Inventive concept 40,
   wherein the target arm includes a central housing, which includes the primary target pivot point and the secondary target pivot point,
   wherein the locking mechanism includes a locking screw having a distal end arranged to come in contact with a proximal portion of the primary sight arm at a location proximal to the primary target pivot point, and
   wherein such contact prevents motion of the sight away from the target-stabilization unit.
Inventive concept 42. The apparatus according to any one of Inventive concepts 1-20, wherein a first distance between the primary target pivot point and the secondary target pivot point is not equal to a second distance between the primary sight pivot point and the secondary sight pivot point.
Inventive concept 43. The apparatus according to Inventive concept 42, wherein (a) the lesser of the first and the second distances is less than 90% of (b) the greater of the first and the second distances.
Inventive concept 44. The apparatus according to Inventive concept 43, wherein (a) the lesser of the first and the second distances is less than 75% of (b) the greater of the first and the second distances.
Inventive concept 45. The apparatus according to Inventive concept 44, wherein (a) the lesser of the first and the second distances is less than 66% of (b) the greater of the first and the second distances.
Inventive concept 46. The apparatus according to Inventive concept 42, wherein the first distance is less than the second distance.
Inventive concept 47. The apparatus according to any one of Inventive concepts 1-20, wherein a third line defined by the primary sight pivot point and the secondary sight pivot point is parallel to or forms a sight-target-axis angle of less than 5 degrees with the sight target axis over the entire range of angles.
Inventive concept 48. The apparatus according to Inventive concept 47, wherein the sight-target-axis angle is less than 2.5 degrees over the entire range of angles.
Inventive concept 49. The apparatus according to Inventive concept 47, wherein the third line defined by the primary sight pivot point and the secondary sight pivot point is parallel to the sight target axis over the entire range of angles.
Inventive concept 50. The apparatus according to any one of Inventive concepts 1-20, for use with an external mounting device having an interface, wherein the surgical cutting tool guide is shaped so as to define one or more interfaces configured to be coupled to the interface of the external mounting device.

There is further provided, in accordance with an Inventive concept 51 of the present invention, apparatus including a surgical cutting tool guide for use with a surgical cutting tool, the surgical cutting tool guide including:
a target arm;
a primary sight arm, which is pivotably attached to the target arm such that the primary sight arm and the target arm are rotatable with respect to each other about a primary target pivot point, so as to permit relative movement between the primary sight arm and the target arm;
a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm; and
a sight, which includes a sight housing, which (a) defines a sight passage for aligning the surgical cutting tool with a sight target axis, and (b) is attached to:
   the primary sight arm such that the sight housing and the primary sight arm are rotatable with respect to each other about a primary sight pivot point, and
   the secondary sight arm such that the sight housing and the secondary sight arm are rotatable with respect to each other about a secondary sight pivot point that does not coincide with the primary sight pivot point,
wherein the primary target pivot point, the secondary target pivot point, the primary sight pivot point, and the secondary sight pivot point (a) are arranged a quadrilateral and (b) are not arranged as a parallelogram over an entire range of angles that extends at least between 11 degrees and 22 degrees, wherein the angles correspond to respective different orientations of the primary sight arm with respect to the target arm and are measured between (a) a first line that is defined by a target point and the primary target pivot point and (b) a second line that (i) intersects the first line and (ii) intersects and is perpendicular to the sight target axis.
Inventive concept 52. The apparatus according to Inventive concept 51,
   wherein the quadrilateral has a first angle at the primary sight pivot point and a second angle at the secondary target pivot point, and
   wherein a difference between the first and the second angles equals at least 75 degrees over the entire range of angles between the first and the second lines.

There is still further provided, in accordance with an Inventive concept 53 of the present invention, apparatus including a surgical cutting tool guide for use with a surgical cutting tool, the surgical cutting tool guide including:
a target arm;
a primary sight arm, which is pivotably attached to the target arm such that the primary sight arm and the target arm are rotatable with respect to each other about a primary target pivot point, so as to permit relative movement between the primary sight arm and the target arm;
a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm; and
a sight, which includes a sight housing, which (a) defines a sight passage for aligning the surgical cutting tool with a sight target axis, and (b) is attached to:
   the primary sight arm such that the sight housing and the primary sight arm are rotatable with respect to each other about a primary sight pivot point, and
   the secondary sight arm such that the sight housing and the secondary sight arm are rotatable with respect to each other about a secondary sight pivot point that does not coincide with the primary sight pivot point,
wherein a first distance between the primary target pivot point and the secondary target pivot point is not equal to a second distance between the primary sight pivot point and the secondary sight pivot point.
Inventive concept 54. The apparatus according to Inventive concept 53, wherein (a) the lesser of the first and the second distances is less than 90% of (b) the greater of the first and the second distances.
Inventive concept 55. The apparatus according to Inventive concept 54, wherein (a) the lesser of the first and the second distances is less than 75% of (b) the greater of the first and the second distances.
Inventive concept 56. The apparatus according to Inventive concept 55, wherein (a) the lesser of the first and the second distances is less than 66% of (b) the greater of the first and the second distances.
Inventive concept 57. The apparatus according to Inventive concept 53, wherein the first distance is less than the second distance.

There is additionally provided, in accordance with an Inventive concept 58 of the present invention, apparatus including a surgical cutting tool guide for use with a surgical cutting tool, the surgical cutting tool guide including:
a target arm;
a primary sight arm, which is pivotably attached to the target arm such that the primary sight arm and the target arm are rotatable with respect to each other about a primary target pivot point, so as to permit relative movement between the primary sight arm and the target arm;
a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm; and
a sight, which includes a sight housing, which (a) defines a sight passage for aligning the surgical cutting tool with a sight target axis, and (b) is attached to:
   the primary sight arm such that the sight housing and the primary sight arm are rotatable with respect to each other about a primary sight pivot point, and
   the secondary sight arm such that the sight housing and the secondary sight arm are rotatable with respect to each other about a secondary sight pivot point that does not coincide with the primary sight pivot point,
wherein a third line defined by the primary sight pivot point and the secondary sight pivot point is parallel to or forms a sight-target-axis angle of less than 5 degrees with the sight target axis over an entire range of angles that extends at least between 11 degrees and 22 degrees, wherein the angles correspond to respective different orientations of the primary sight arm with respect to the target arm and are measured between (a) a first line that is defined by a target point and the primary target pivot point and (b) a second line that (i) intersects the first line and (ii) intersects and is perpendicular to the sight target axis.
Inventive concept 59. The apparatus according to Inventive concept 58, wherein the sight-target-axis angle is less than 2.5 degrees over the entire range of angles.
Inventive concept 60. The apparatus according to Inventive concept 58, wherein the third line defined by the primary sight pivot point and the secondary sight pivot point is parallel to the sight target axis over the entire range of angles.

There is yet additionally provided, in accordance with an Inventive concept 61 of the present invention, apparatus including a surgical cutting tool guide for use with a surgical cutting tool, the surgical cutting tool guide including:
a target arm;
a primary sight arm, which is pivotably attached to the target arm such that the primary sight arm and the target arm are rotatable with respect to each other about a primary target pivot point, so as to permit relative movement between the primary sight arm and the target arm;
a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm;
a sight, which includes a sight housing, which (a) defines a sight passage for aligning the surgical cutting tool with a sight target axis, and (b) is attached to:
   the primary sight arm such that the sight housing and the primary sight arm are rotatable with respect to each other about a primary sight pivot point, and
   the secondary sight arm such that the sight housing and the secondary sight arm are rotatable with respect to each other about a secondary sight pivot point that does not coincide with the primary sight pivot point;
a target-stabilization unit, which is (a) disposed at a distal end of the target arm, (b) configured to stabilize a target point with respect to a body of a patient, and (c) shaped so as to define a target housing that is disposed around at least 180 degrees of the target point; and
joints that facilitate relative rotation of the target housing about:
   a first line over a first entire range of angles that extends at least 90 degrees, wherein the first line is defined by the target point and the primary target pivot point, and
   a line perpendicular to both the first line and the sight target axis over a second entire range of angles that extends at least 90 degrees, and
wherein the target point moves, with respect to the target housing, by less than 2% of the target axis distance during rotation of the target housing over the first entire range of angles and the second entire range of angles.
Inventive concept 62. The apparatus according to Inventive concept 61,
   wherein the joints additionally facilitate relative rotation of an element of the target housing about the sight target axis over a third entire range of angles that extends at least 90 degrees, and
   wherein the target point moves, with respect to the target housing, by less than 2% of the target axis distance during rotation of the target housing over the first entire range of angles, the second entire range of angles, and the third entire range of angles.
Inventive concept 63. The apparatus according to Inventive concept 61, wherein the target housing is shaped so as to define a surface facing the target point.
Inventive concept 64. The apparatus according to Inventive concept 61, wherein the target-stabilization unit and the sight are arranged to grasp a bony part of the body therebetween.
Inventive concept 65. The apparatus according to Inventive concept 64, wherein the surgical cutting tool guide further includes a locking mechanism, which is configured to lock the sight with respect to the target-stabilization unit.
Inventive concept 66. The apparatus according to any one of Inventive concepts 61-65, wherein the target housing is disposed around at least 270 degrees of the target point.
Inventive concept 67. The apparatus according to Inventive concept 66, wherein the target housing entirely surrounds the target point.
Inventive concept 68. The apparatus according to Inventive concept 67, wherein the target housing is shaped so as to define an annular surface facing the target point.
Inventive concept 69. The apparatus according to Inventive concept 68, wherein the annular surface is circular or polygonal.
Inventive concept 70. The apparatus according to Inventive concept 67, wherein the target point is centered in the target housing.

There is also provided, in accordance with an Inventive concept 71 of the present invention, a method for drilling a bore into a zygomatic bone for anchoring a zygomatic implant, the method including:
providing a surgical cutting tool guide for use with a surgical cutting tool, the surgical cutting tool guide including (i) a target arm; (ii) a primary sight arm, which is pivotably attached to the target arm such that the primary sight arm and the target arm are rotatable with respect to each other about a primary target pivot point, so as to permit relative movement between the primary sight arm and the target arm; (iii) a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm; (iv) a sight, which includes a sight housing, which (a) defines a sight passage for aligning the surgical cutting tool with a sight target axis, and (b) is attached to (A) the primary sight arm such that the sight housing and the primary sight arm are rotatable with respect to each other about a primary sight pivot point, and (B) the secondary sight arm such that the sight housing and the secondary sight arm are rotatable with respect to each other about a secondary sight pivot point that does not coincide with the primary sight pivot point; and (v) a target-stabilization unit, which is disposed at a distal end of the target arm, and is configured to stabilize a target point with respect to the zygomatic bone,
   wherein the surgical cutting tool guide is arranged to automatically and non-electrically maintain the sight target axis oriented to pass within an offset distance of the target point over an entire range of angles that extends at least between 11 degrees and 22 degrees, wherein the angles correspond to respective different orientations of the primary sight arm with respect to the target arm and are measured between (a) a first line that is defined by the target point and the primary target pivot point and (b) a second line that (i) intersects the first line and (ii) intersects and is perpendicular to the sight target axis, and
   wherein the offset distance equals less than 5% of a target axis distance measured between the target point and the primary target pivot point, such that the sight directs the sight passage toward the target point over the entire range of angles;
inserting the sight and portions of the primary and the secondary sight arms through a mouth into an oral cavity;
positioning the target point at or near a malar point and stabilizing the target point with respect to the zygomatic bone, by placing the target-stabilization unit against skin of a cheek;
positioning the sight near a desired implantation site of a maxilla;
pushing the sight tightly against the implantation site by bringing the sight closer to the target-stabilization unit, by bringing the primary and the secondary sight arms closer to the target arm;
advancing the surgical cutting tool through the sight passage, toward the target point; and
drilling the bore using the surgical cutting tool.
Inventive concept 72. The method according to Inventive concept 71,
   wherein the target-stabilization unit is shaped so as to define a target housing that is disposed around at least 180 degrees of the target point, and
   wherein placing the target-stabilization unit against the skin of the cheek includes disposing the target housing against the skin partially or entirely around the malar point.
Inventive concept 73. The method according to Inventive concept 71,
   wherein the surgical cutting tool guide further includes a locking mechanism, which is configured to lock the sight with respect to the target-stabilization unit, and
   wherein the method further includes using the locking mechanism to lock the sight with respect to the target-stabilization unit, thereby maintaining the sight clamped tightly against the implantation site and the target-stabilization unit tightly against the skin of the cheek at or near the malar point, and the sight at a fixed position and orientation with respect to the zygomatic bone.

There is further provided, in accordance with an Inventive concept 74 of the present invention, a method for drilling a bore into a zygomatic bone for anchoring a zygomatic implant, the method including:
inserting a sight through a mouth into an oral cavity;
outside the oral cavity, positioning a target point at or near a malar point;
positioning the sight near a desired implantation site of a maxilla, wherein the sight includes a sight housing that defines a sight passage for aligning a surgical cutting tool;
orienting the sight passage to point toward the target point;
advancing the surgical cutting tool through the sight passage, toward the target point; and
drilling the bore using the surgical cutting tool.
Inventive concept 75. The method according to Inventive concept 74, wherein positioning the target point includes stabilizing the target point with respect to the zygomatic bone.
Inventive concept 76. The method according to Inventive concept 75, wherein stabilizing the target point with respect to the zygomatic bone includes placing a target-stabilization unit against skin of a cheek.
Inventive concept 77. The method according to Inventive concept 76, wherein positioning the sight near the desired implantation site includes pushing the sight tightly against the desired implantation site by bringing the sight closer to the target-stabilization unit.
Inventive concept 78. The method according to Inventive concept 76, wherein placing the target-stabilization unit against the skin of the cheek includes bringing the sight and the target-stabilization unit closer to each other.
Inventive concept 79. The method according to Inventive concept 77,
   wherein the target-stabilization unit is shaped so as to define a target housing that is disposed around at least 180 degrees of the target point, and
   wherein placing the target-stabilization unit against the skin of the cheek includes disposing the target housing against the skin partially or entirely around the malar point.
Inventive concept 80. The method according to Inventive concept 74, wherein positioning the sight near the desired implantation site includes pushing the sight tightly against the desired implantation site.
Inventive concept 81. The method according to Inventive concept 74, wherein orienting the sight passage to point toward the target point includes orienting the sight passage to point toward the target point using a surgical cutting tool guide that is arranged to automatically and non-electrically orient the sight passage to point toward the target point, the surgical cutting tool guide including the sight.

There is still further provided, in accordance with an Inventive concept 82 of the present invention, apparatus including a surgical cutting tool guide for use with a surgical cutting tool, the surgical cutting tool guide including:
a target arm;
a target-stabilization unit, which is (a) disposed at a distal end of the target arm, (b) configured to stabilize a target point with respect to a body of a patient, and (c) shaped so as to define a target housing that is disposed around at least 180 degrees of the target point;
one or more sight arms;
a sight, which is attached to the one or more sight arms, and which includes a sight housing, which defines a sight passage for aligning the surgical cutting tool with a sight target axis, wherein the surgical cutting tool guide is arranged to permit relative movement between the sight and the target arm; and
joints that facilitate relative rotation of the target housing about:
   a target-arm axis over a first entire range of angles that extends at least 90 degrees, and
   a line perpendicular to both the target-arm axis and the sight target axis over a second entire range of angles that extends at least 90 degrees,
wherein the target point moves, with respect to the target housing, by less than 2 mm during rotation of the target housing over the first entire range of angles and the second entire range of angles,
wherein the surgical cutting tool guide is arranged to automatically and non-electrically maintain the sight target axis oriented to pass within an offset distance of the target point, and
wherein the offset distance equals less than 2.5 mm, such that the sight directs the sight passage toward the target point.
Inventive concept 83. The apparatus according to Inventive concept 82, wherein the offset distance equals less than 1.5 mm.
Inventive concept 84. The apparatus according to Inventive concept 83, wherein the offset distance equals less than 1 mm.
Inventive concept 85. The apparatus according to Inventive concept 82,
   wherein the joints additionally facilitate relative rotation of an element of the target housing about the sight target axis over a third entire range of angles that extends at least 90 degrees, and
   wherein the target point moves, with respect to the target housing, by less than 2 mm during rotation of the target housing over the first entire range of angles, the second entire range of angles, and the third entire range of angles.
Inventive concept 86. The apparatus according to Inventive concept 82, wherein the target housing is shaped so as to define a surface facing the target point.
Inventive concept 87. The apparatus according to Inventive concept 82, wherein the target-stabilization unit and the sight are arranged to grasp a bony part of the body therebetween.
Inventive concept 88. The apparatus according to Inventive concept 87, wherein the surgical cutting tool guide further includes a locking mechanism, which is configured to lock the sight with respect to the target-stabilization unit.
Inventive concept 89. The apparatus according to any one of Inventive concepts 82-88, wherein the target housing is disposed around at least 270 degrees of the target point.
Inventive concept 90. The apparatus according to Inventive concept 89, wherein the target housing entirely surrounds the target point.
Inventive concept 91. The apparatus according to Inventive concept 90, wherein the target housing is shaped so as to define an annular surface facing the target point.
Inventive concept 92. The apparatus according to Inventive concept 91, wherein the annular surface is circular or polygonal.
Inventive concept 93. The apparatus according to Inventive concept 90, wherein the target point is centered in the target housing.
Inventive concept 94. The apparatus according to any one of Inventive concepts 82-88,
   wherein the surgical cutting tool guide further includes a main body, which includes an elongate support structure,
   wherein a proximal portion of the target arm is attached to the main body,
   wherein the one or more sight arms are slidably attached to the elongate support structure, such that the one or more sight arms can move linearly and assume a plurality of axial positions along the elongate support structure, thereby permitting the relative movement between the sight and the target arm, and
   wherein the surgical cutting tool guide is arranged to automatically and non-electrically maintain the sight target axis oriented to pass within the offset distance of the target point at all of the axial positions of the one or more sight arms along the elongate support structure.
Inventive concept 95. The apparatus according to Inventive concept 94, wherein the main body includes a mechanical control element that is disposed remotely from the one or more sight arms, and is configured to mechanically and non-electrically linearly move the one or more sight arms along the elongate support structure.
Inventive concept 96. The apparatus according to any one of Inventive concepts 82-88,
   wherein the one or more sight arms include:
      a primary sight arm, which is pivotably attached to the target arm such that the primary sight arm and the target arm are rotatable with respect to each other about a primary target pivot point, so as to permit relative movement between the primary sight arm and the target arm; and
      a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm, and
   wherein the sight is attached to:
      the primary sight arm such that the sight housing and the primary sight arm are rotatable with respect to each other about a primary sight pivot point, and
      the secondary sight arm such that the sight housing and the secondary sight arm are rotatable with respect to each other about a secondary sight pivot point that does not coincide with the primary sight pivot point.
Inventive concept 97. The apparatus according to any one of Inventive concepts 82-88, wherein the cutting tool is a drill bit, wherein the surgical cutting tool guide is a surgical drill guide, and wherein the sight passage is arranged to guide the surgical drill bit along the sight target axis.
Inventive concept 98. The apparatus according to any one of Inventive concepts 82-88, wherein the cutting tool is a milling bit, wherein the surgical cutting tool guide is a surgical milling guide, and wherein the sight passage is arranged to guide the surgical milling bit along the sight target axis.
Inventive concept 99. The apparatus according to any one of Inventive concepts 82-88, wherein the cutting tool is a laser cutting tool, wherein the surgical cutting tool guide is a surgical laser cutting tool guide, and wherein the sight passage is arranged to align the laser cutting tool along the sight target axis.
Inventive concept 100. The apparatus according to any one of Inventive concepts 82-88, wherein the cutting tool is an ultrasonic cutting tool, wherein the surgical cutting tool guide is a surgical ultrasonic cutting tool guide, and wherein the sight passage is arranged to align the ultrasonic cutting tool with the sight target axis.

There is additionally provided, in accordance with an Inventive concept 101 of the present invention, a method for drilling a bore into a zygomatic bone for anchoring a zygomatic implant, the method including:
providing a surgical cutting tool guide for use with a surgical cutting tool, the surgical cutting tool guide including (i) a target arm; (ii) one or more sight arms, which are moveable with respect to the target arm; (iii) a sight, which includes a sight housing, which (a) defines a sight passage for aligning the surgical cutting tool with a sight target axis, and (b) is attached to the one or more sight arms; and (iv) a target-stabilization unit, which is disposed at a distal end of the target arm, and is configured to stabilize a target point with respect to the zygomatic bone,
   wherein the one or more sight arms are moveable with respect to the target arm, thereby permitting relative movement between the sight and the target arm,
   wherein the surgical cutting tool guide is arranged to automatically and non-electrically maintain the sight target axis oriented to pass within an offset distance of the target point, and
   wherein the offset distance equals less than 2.5 mm, such that the sight directs the sight passage toward the target point;
inserting the sight and portions of the one or more sight arms through a mouth into an oral cavity;
positioning the target point at or near a malar point and stabilizing the target point with respect to the zygomatic bone, by placing the target-stabilization unit against skin of a cheek;
positioning the sight near a desired implantation site of a maxilla; pushing the sight tightly against the implantation site by bringing the sight closer to the target-stabilization unit, by bringing the one or more sight arms closer to the target arm;
advancing the surgical cutting tool through the sight passage, toward the target point; and
drilling the bore using the surgical cutting tool.
Inventive concept 102. The method according to Inventive concept 101,
   wherein the target-stabilization unit is shaped so as to define a target housing that is disposed around at least 180 degrees of the target point, and
   wherein placing the target-stabilization unit against the skin of the cheek includes disposing the target housing against the skin partially or entirely around the malar point.
Inventive concept 103. The method according to Inventive concept 102,
   wherein the surgical cutting tool guide further includes joints that facilitate relative rotation of the target housing about: (a) a target-arm axis over a first entire range of angles that extends at least 90 degrees, and (b) a line perpendicular to both the target-arm axis and the sight target axis over a second entire range of angles that extends at least 90 degrees,
   wherein the target point moves, with respect to the target housing, by less than 2 mm during rotation of the target housing over the first entire range of angles and the second entire range of angles,
104. The method according to Inventive concept 103,
   wherein the joints additionally facilitate relative rotation of an element of the target housing about the sight target axis over a third entire range of angles that extends at least 90 degrees, and
   wherein the target point moves, with respect to the target housing, by less than 2 mm during rotation of the target housing over the first entire range of angles, the second entire range of angles, and the third entire range of angles.
Inventive concept 105. The method according to Inventive concept 102, wherein the target housing is shaped so as to define a surface facing the target point.
Inventive concept 106. The method according to Inventive concept 102, wherein the target housing is disposed around at least 270 degrees of the target point.
Inventive concept 107. The method according to Inventive concept 106, wherein the target housing entirely surrounds the target point.
Inventive concept 108. The method according to Inventive concept 107, wherein the target housing is shaped so as to define an annular surface facing the target point.
Inventive concept 109. The method according to Inventive concept 108, wherein the annular surface is circular or polygonal.
Inventive concept 110. The method according to Inventive concept 107, wherein the target point is centered in the target housing.
Inventive concept 111. The method according to Inventive concept 101,
   wherein the surgical cutting tool guide further includes a main body, which includes an elongate support structure,
   wherein a proximal portion of the target arm is attached to the main body,
   wherein the one or more sight arms are slidably attached to the elongate support structure, such that the one or more sight arms can move linearly and assume a plurality of axial positions along the elongate support structure, thereby permitting the relative movement between the sight and the target arm,
   wherein the surgical cutting tool guide is arranged to automatically and non-electrically maintain the sight target axis oriented to pass within the offset distance of the target point at all of the axial positions of the one or more sight arms along the elongate support structure, and
   wherein pushing the sight tightly against the implantation site by bringing the sight closer to the target-stabilization unit includes sliding the one or more sight arms along the elongate support structure toward the target arm.
Inventive concept 112. The method according to Inventive concept 101,
   wherein the one or more sight arms include:
      a primary sight arm, which is pivotably attached to the target arm such that the primary sight arm and the target arm are rotatable with respect to each other about a primary target pivot point, so as to permit relative movement between the primary sight arm and the target arm;
      a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm,
   wherein the sight is attached to:
      the primary sight arm such that the sight housing and the primary sight arm are rotatable with respect to each other about a primary sight pivot point, and
      the secondary sight arm such that the sight housing and the secondary sight arm are rotatable with respect to each other about a secondary sight pivot point that does not coincide with the primary sight pivot point, and
   wherein pushing the sight tightly against the implantation site by bringing the sight closer to the target-stabilization unit includes bringing the primary and the secondary sight arms closer to the target arm.

There is yet additionally provided, in accordance with an Inventive concept 113 of the present invention, apparatus including a surgical cutting tool guide for use with a surgical cutting tool, the surgical cutting tool guide including:
a main body, which is shaped so as to define an elongate support structure;
a target arm, a proximal portion of which is attached to the main body;
a target-stabilization unit, which is disposed at a distal end of the target arm, and is configured to stabilize a target point with respect to a body of a patient;
a sight, which includes a sight housing, which defines a sight passage for aligning the surgical cutting tool with a sight target axis; and
a sight arm, which is attached to the sight and slidably attached to the elongate support structure, such that the sight arm can move linearly and assume a plurality of axial positions along the elongate support structure, thereby permitting relative movement between the sight and the target arm,
wherein the surgical cutting tool guide is arranged to automatically and non-electrically maintain the sight target axis oriented to pass within an offset distance of the target point at all of the axial positions of the sight arm along the elongate support structure, and
wherein the offset distance equals less than 2.5 mm, such that the sight directs the sight passage toward the target point at all of the axial positions of the sight arm along the elongate support structure.
Inventive concept 114. The apparatus according to Inventive concept 113, wherein the offset distance equals less than 1.5 mm.
Inventive concept 115. The apparatus according to Inventive concept 113, wherein the main body includes a mechanical control element that is disposed remotely from the sight arm, and is configured to mechanically and non-electrically linearly move the sight arm along the elongate support structure.
Inventive concept 116. The apparatus according to Inventive concept 113, wherein the target-stabilization unit is shaped so as to define a target housing that is disposed around at least 180 degrees of the target point.
Inventive concept 117. The apparatus according to Inventive concept 116, wherein the target housing is shaped so as to define a surface facing the target point.
Inventive concept 118. The apparatus according to Inventive concept 116, wherein the target housing is disposed around at least 270 degrees of the target point.
Inventive concept 119. The apparatus according to Inventive concept 118, wherein the target housing entirely surrounds the target point.
Inventive concept 120. The apparatus according to Inventive concept 119, wherein the target housing is shaped so as to define an annular surface facing the target point.
Inventive concept 121. The apparatus according to Inventive concept 120, wherein the annular surface is circular or polygonal.
Inventive concept 122. The apparatus according to Inventive concept 119, wherein the target point is centered in the target housing.
Inventive concept 123. The apparatus according to Inventive concept 116, wherein the surgical cutting tool guide includes a joint that facilitates relative rotation of the target housing about an axis of the target arm over an entire range of angles that extends at least 90 degrees, and wherein the target point moves, with respect to the target housing, by less than 2 mm during rotation of the target housing over the entire range of angles.
Inventive concept 124. The apparatus according to Inventive concept 116, wherein the surgical cutting tool guide includes a joint that facilitates relative rotation of the target housing about a line perpendicular to both an axis of the target arm and the sight target axis over an entire range of angles that extends at least 90 degrees, and wherein the target point moves, with respect to the target housing, by less than 2 mm during rotation of the target housing over the entire range of angles.
Inventive concept 125. The apparatus according to Inventive concept 116, wherein the surgical cutting tool guide includes a joint that facilitates relative rotation of an element of the target housing about the sight target axis over an entire range of angles that extends at least 90 degrees, and wherein the target point moves, with respect to the target housing, by less than 2 mm during rotation of the target housing over the entire range of angles.
Inventive concept 126. The apparatus according to Inventive concept 116,
   wherein the surgical cutting tool guide includes joints that facilitate relative rotation of the target housing about:
      an axis of the target arm over a first entire range of angles that extends at least 90 degrees, and
      a line perpendicular to both the axis of the target arm and the sight target axis over a second entire range of angles that extends at least 90 degrees, and
   wherein the target point moves, with respect to the target housing, by less than 2 mm during rotation of the target housing over the first entire range of angles and the second entire range of angles.
Inventive concept 127. The apparatus according to Inventive concept 116,
   wherein the surgical cutting tool guide includes joints that facilitate relative rotation of:
      the target housing about an axis of the target arm over a first entire range of angles that extends at least 90 degrees,
      the target housing about a line perpendicular to both the axis of the target arm and the sight target axis over a second entire range of angles that extends at least 90 degrees, and
      an element of the target housing about the sight target axis over a third entire range of angles that extends at least 90 degrees, and
   wherein the target point moves, with respect to the target housing, by less than 2 mm during rotation of the target housing over the first entire range of angles, the second entire range of angles, and the third entire range of angles.
Inventive concept 128. The apparatus according to Inventive concept 113, wherein the target-stabilization unit and the sight are arranged to grasp a bony part of the body therebetween.
Inventive concept 129. The apparatus according to Inventive concept 128, wherein the surgical cutting tool guide further includes a locking mechanism, which is configured to lock the sight with respect to the target-stabilization unit.
Inventive concept 130. The apparatus according to any one of Inventive concepts 113-129, wherein the cutting tool is a drill bit, wherein the surgical cutting tool guide is a surgical drill guide, and wherein the sight passage is arranged to guide the surgical drill bit along the sight target axis.
Inventive concept 131. The apparatus according to any one of Inventive concepts 113-129, wherein the cutting tool is a milling bit, wherein the surgical cutting tool guide is a surgical milling guide, and wherein the sight passage is arranged to guide the surgical milling bit along the sight target axis.
Inventive concept 132. The apparatus according to any one of Inventive concepts 113-129, wherein the cutting tool is a laser cutting tool, wherein the surgical cutting tool guide is a surgical laser cutting tool guide, and wherein the sight passage is arranged to align the laser cutting tool along the sight target axis.
Inventive concept 133. The apparatus according to any one of Inventive concepts 113-129, wherein the cutting tool is an ultrasonic cutting tool, wherein the surgical cutting tool guide is a surgical ultrasonic cutting tool guide, and wherein the sight passage is arranged to align the ultrasonic cutting tool with the sight target axis.

There is also provided, in accordance with an Inventive concept 134 of the present invention, a method for drilling a bore into a zygomatic bone for anchoring a zygomatic implant, the method including:
providing a surgical cutting tool guide for use with a surgical cutting tool, the surgical cutting tool guide including (a) a main body, which is shaped so as to define an elongate support structure; (b) a target arm, a proximal portion of which is attached to the main body; (c) a target-stabilization unit, which is disposed at a distal end of the target arm, and is configured to stabilize a target point with respect to a body of a patient; (d) a sight, which includes a sight housing, which defines a sight passage for aligning the surgical cutting tool with a sight target axis, and (e) a sight arm, which is attached to the sight arm and slidably attached to the elongate support structure, such that the sight arm can move linearly and assume a plurality of axial positions along the elongate support structure, thereby permitting relative movement between the sight and the target arm,
   wherein the surgical cutting tool guide is arranged to automatically and non-electrically maintain the sight target axis oriented to pass within an offset distance of the target point at all of the axial positions of the sight arm along the elongate support structure, and
   wherein the offset distance equals less than 2.5 mm, such that the sight directs the sight passage toward the target point at all of the axial positions of the sight arm along the elongate support structure;
inserting the sight and a portion of the sight arm through a mouth into an oral cavity;
positioning the target point at or near a malar point and stabilizing the target point with respect to the zygomatic bone, by placing the target-stabilization unit against skin of a cheek;
positioning the sight near a desired implantation site of a maxilla;
pushing the sight tightly against the implantation site by bringing the sight closer to the target-stabilization unit, by bringing the sight arm closer to the target arm;
advancing the surgical cutting tool through the sight passage, toward the target point; and
drilling the bore using the surgical cutting tool.
Inventive concept 135. The method according to Inventive concept 134,
   wherein the target-stabilization unit is shaped so as to define a target housing that is disposed around at least 180 degrees of the target point, and
   wherein placing the target-stabilization unit against the skin of the cheek includes disposing the target housing against the skin partially or entirely around the malar point.
Inventive concept 136. The method according to Inventive concept 134,
   wherein the surgical cutting tool guide further includes a locking mechanism, which is configured to lock the sight with respect to the target-stabilization unit, and
   wherein the method further includes using the locking mechanism to lock the sight with respect to the target-stabilization unit, thereby maintaining the sight clamped tightly against the implantation site and the target-stabilization unit tightly against the skin of the cheek at or near the malar point, and the sight at a fixed position and orientation with respect to the zygomatic bone.

The present invention will be more fully understood from the following detailed description of embodiments thereof, taken together with the drawings, in which:

### BRIEF DESCRIPTION OF THE DRAWINGS

FIG. 1 is a schematic illustration of a surgical cutting tool guide, in accordance with an application of the present invention;

FIG. 2 is a schematic illustration of a portion of the surgical cutting tool guide of FIG. 1, in accordance with an application of the present invention;

FIGS. 3A and 3B are schematic illustrations of the surgical cutting tool guide of FIG. 1 with respective different orientations of a primary sight arm with respect to a target arm thereof, in accordance with an application of the present invention;

FIG. 4 is a schematic illustration of a target-stabilization unit of the surgical cutting tool guide of FIG. 1, in accordance with an application of the present invention;

FIGS. 5A-C are schematic illustrations of the use of the surgical cutting tool guide of FIG. 1 for drilling a bore into a zygomatic bone for anchoring a zygomatic implant, in accordance with an application of the present invention;

FIG. 6 is a schematic illustration of another surgical cutting tool guide, in accordance with an application of the present invention;

FIG. 7 is a schematic illustration of yet another surgical cutting tool guide, in accordance with an application of the present invention;

FIGS. 8A-B are schematic illustrations of two axial positions of a lead screw of the surgical cutting tool guide of FIG. 7 and the resulting relative positions of primary and secondary sight arms with respect to a target point, in accordance with an application of the present invention;

FIGS. 9A-C are schematic illustrations of a sight, in accordance with an application of the present invention;

FIG. 10 is a schematic illustration of a still another surgical cutting tool guide, in accordance with an application of the present invention;

FIGS. 11A-C are schematic illustrations of respective configurations of the surgical cutting tool guide of FIG. 10, in accordance with respective applications of the present invention;

FIGS. 12A-B are schematic illustration of yet another surgical cutting tool guide, in accordance with an application of the present invention;

FIG. 13 is a schematic illustration of a target-stabilization unit of the surgical cutting tool guide of FIGS. 12A-B, in accordance with an application of the present invention;

FIG. 14 is a schematic cross-sectional view of the surgical cutting tool guide of FIGS. 12A-B, in accordance with an application of the present invention; and

FIGS. 15A-E are schematic illustrations of optional components that may be used in conjunction with the surgical cutting tool guides of FIGS. 1, 6, 7, and 12A-B, in accordance with respective applications of the present invention.

### DETAILED DESCRIPTION OF APPLICATIONS

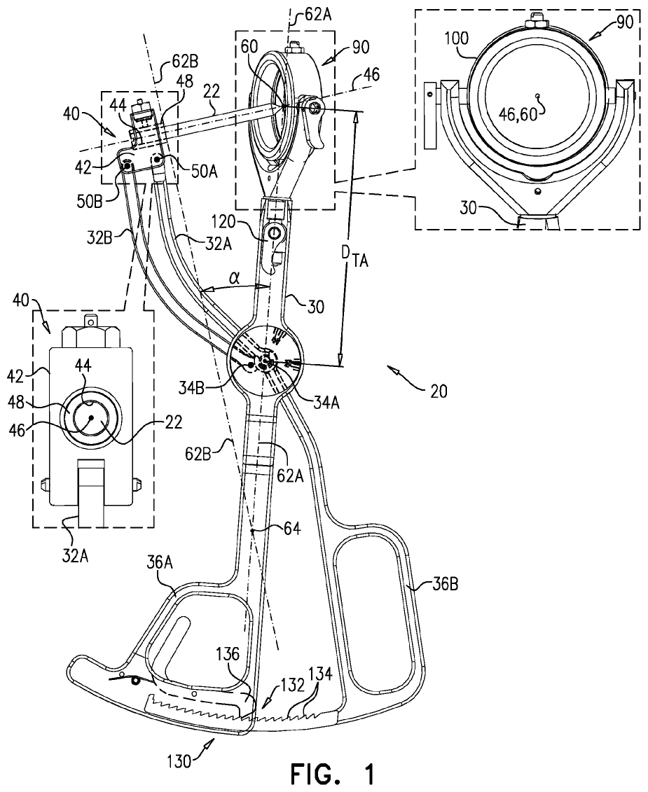

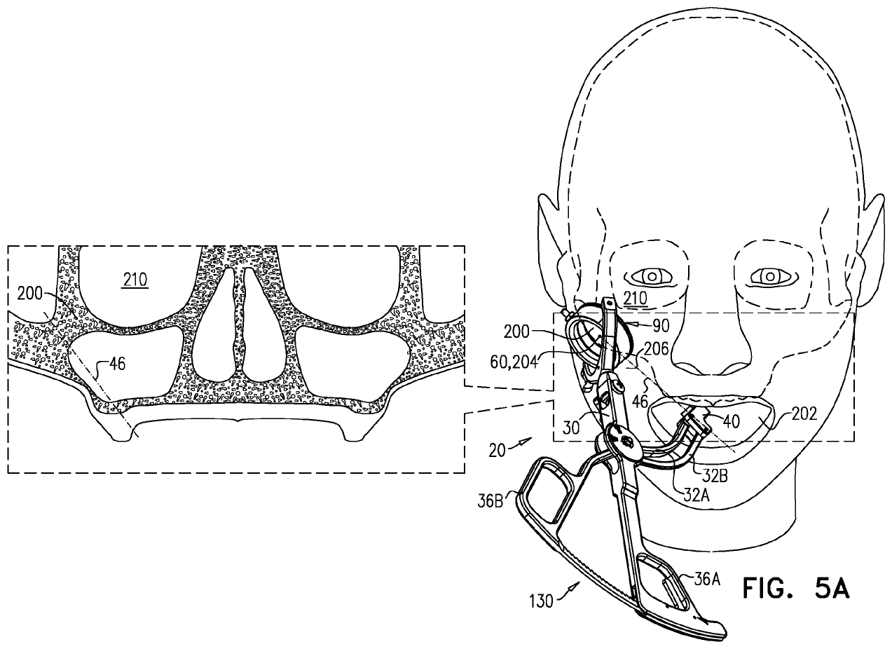

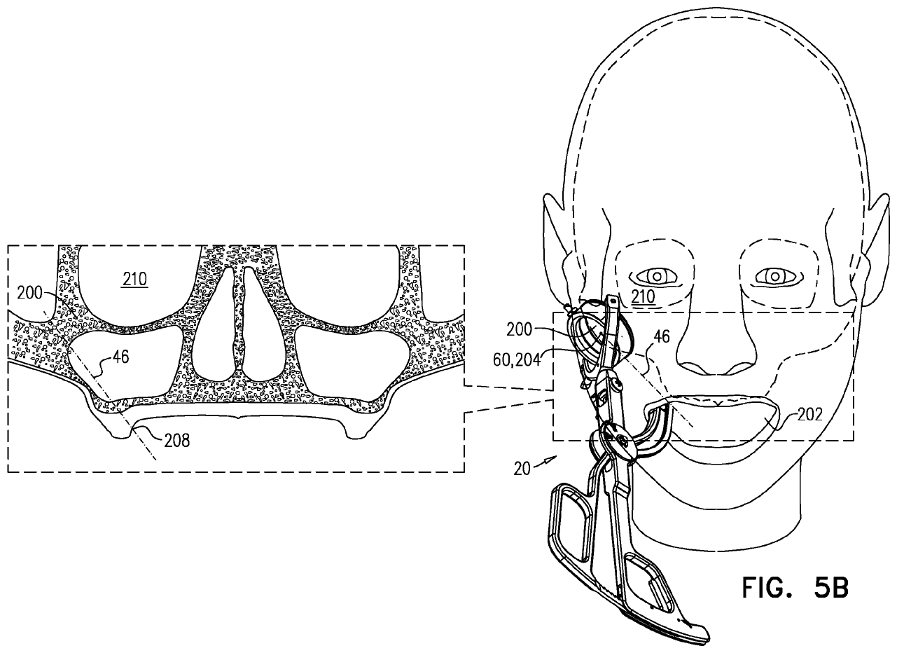

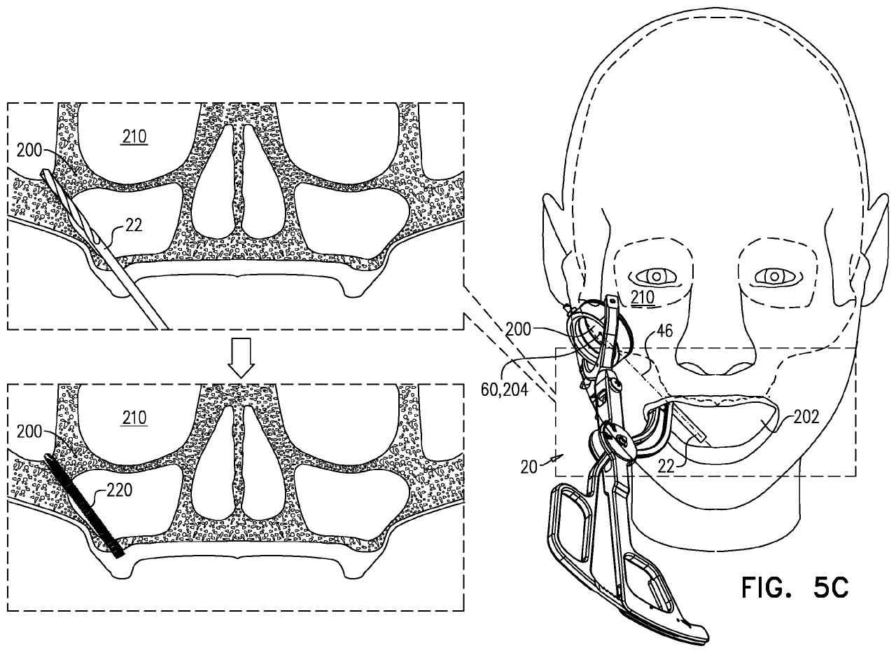

FIG. 1 is a schematic illustration of a surgical cutting tool guide 20, in accordance with an application of the present invention. Surgical cutting tool guide 20 is for use with a surgical cutting tool 22, such as a drill bit, a milling bit, a laser cutting tool, or an ultrasonic cutting tool. Although shown schematically in FIG. 1, surgical cutting tool 22 is not an element of surgical cutting tool guide 20. For some applications, surgical cutting tool guide 20 is used to drill a bore into a zygomatic bone for anchoring a zygomatic implant, such as described hereinbelow with reference to FIGS. 5A-C.

Surgical cutting tool guide 20 typically comprises:
a target arm 30;
a primary sight arm 32A, which is pivotably attached to target arm 30 such that primary sight arm 32A and target arm 30 are rotatable with respect to each other about a primary target pivot point 34A, so as to permit relative movement between primary sight arm 32A and target arm 30; and
a secondary sight arm 32B, which is pivotably attached to target arm 30 such that secondary sight arm 32B and target arm 30 are rotatable with respect to each other about a secondary target pivot point 34B that does not coincide with primary target pivot point 34A, so as to permit relative movement between primary sight arm 32A and target arm 30.

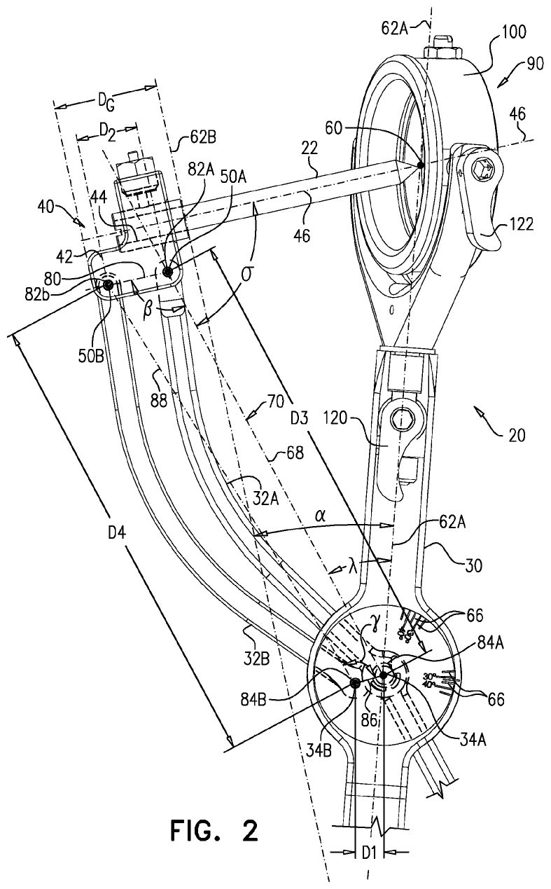

Typically, respective sites of primary target pivot point 34A and secondary target pivot point 34B are fixed with respect to target arm 30 (such as by primary and secondary target pins 84A and 84B, described hereinbelow with reference to FIG. 2). As a result, primary target pivot point 34A and secondary target pivot point 34B cannot move with respect to each other or target arm 30, and primary target pivot point 34A and secondary target pivot point 34B are at a fixed distance from each other (first distance D1, described hereinbelow with reference to FIG. 2). Typically, secondary sight arm 32B runs at least partially alongside primary sight arm 32A. For some applications, target arm 30 and primary sight arm 32A are shaped so as to define respective finger loops 36A and 36B, which facilitate grasping of surgical cutting tool guide 20 by a surgeon, and adjusting the orientation of primary sight arm 32A with respect to target arm 30 (and the orientation of secondary sight arm 32B with respect to target arm 30). Alternatively, for some applications, secondary sight arm 32B is shaped so as to define finger loop 36B.

Surgical cutting tool guide 20 further comprises a sight 40, which comprises a sight housing 42, which typically defines a sight passage 44 for aligning surgical cutting tool 22 with a sight target axis 46. As used in the present application, including in the claims, sight target axis 46 is the set of all centroids of transverse cross-sectional sections of the largest right circular cylinder that can be placed in sight passage 44 along the right circular cylinder. Thus the cross-sectional sections are locally perpendicular to a central longitudinal axis of the right circular cylinder, which runs along the right circular cylinder and sight passage 44. It is to be understood that the right circular cylinder is not a component of surgical cutting tool guide 20, but rather a geometric shape used to define a structural property of the surgical cutting tool guide. Alternatively, for some applications, sight housing 42 does not define sight passage 44, and surgical cutting tool 22 or another tool or device is instead integrated into sight housing 42, rather than inserted into sight passage 44 during a surgical procedure.

For some applications, sight 40 comprises a sight tube 48 (e.g., a guiding sleeve), which is shaped so as to define sight passage 44. Optionally, sight tube 48 is reversibly coupleable to sight housing 42, i.e., exchangeable and/or disposable.

Sight housing 42 is attached to:
primary sight arm 32A such that sight housing 42 and primary sight arm 32A are rotatable with respect to each other about a primary sight pivot point 50A, and
secondary sight arm 32B such that sight housing 42 and secondary sight arm 32B are rotatable with respect to each other about a secondary sight pivot point 50B that does not coincide with primary sight pivot point 50A.

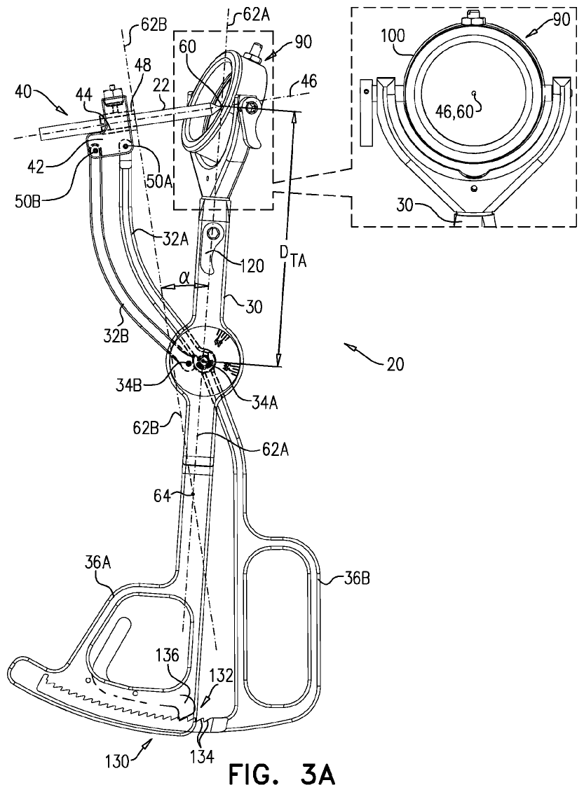

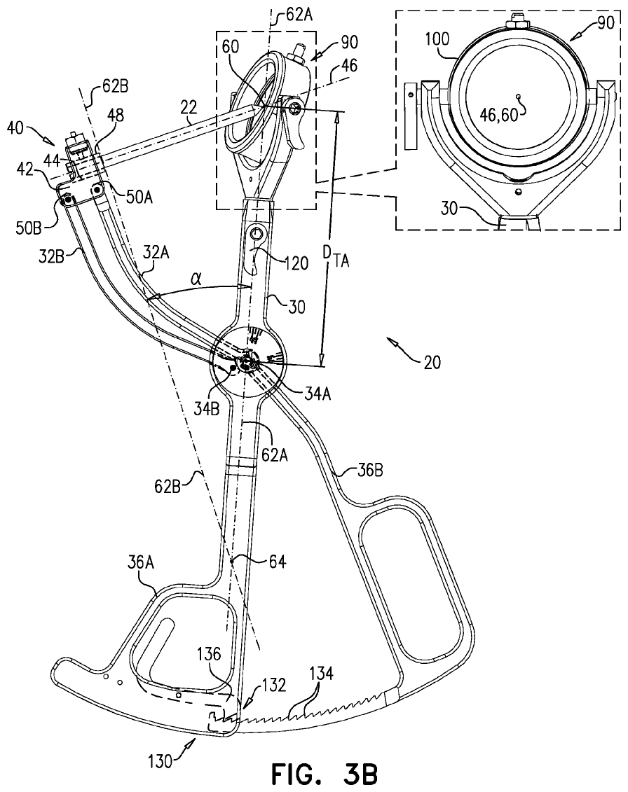

Surgical cutting tool guide 20 is arranged to automatically and non-electrically maintain sight target axis 46 oriented to pass within an offset distance of a target point 60 over an entire range of angles α (alpha) that extends at least between 11 degrees and 22 degrees. Surgical cutting tool guide 20 maintains this orientation by constant relative motion between certain elements of the surgical cutting tool as the arms are moved with respect to one another. The angles α (alpha) correspond to respective different orientations of primary sight arm 32A with respect to target arm 30 (and to respective different orientations of secondary sight arm 32B with respect to target arm 30). The angles are measured between (a) a first line 62A that is defined by target point 60 and primary target pivot point 34A and (b) a second line 62B that (i) intersects first line 62A and (ii) intersects and is perpendicular to sight target axis 46. (In FIG. 1, by way of example, surgical cutting tool guide 20 is shown with primary sight arm 32A oriented with respect to target arm 30 such that angle α (alpha) is approximately 16 degrees.) Typically, an intersection point 64 at which first and second lines 62A and 62B intersect moves along first line 62A and/or second line 62B over the entire range of angles α (alpha), such as described hereinbelow with reference to FIGS. 3A-B.

Typically, the offset distance equals less than (a) 5%, such as less than 3%, e.g., less than 1% (such as less than 0.75%, e.g., less than 0.5%, ideally 0%) of a target axis distance D_{TA} measured between target point 60 and primary target pivot point 34A, and/or (b) less than 2.5 mm, such as less than 1.5 mm, e.g., less than 1 mm (such as less than 0.5 mm, ideally 0 mm), such that sight 40 directs sight passage 44 (and sight target axis 46) toward target point 60 (rather than toward an axis) over the entire range of angles α (alpha). As a result, sight passage 44 guides surgical cutting tool 22 toward target point 60 (within the offset distance) regardless of which angle is set from the entire range of angles α (alpha).

For some applications, the entire range of angles α (alpha) that extends at least between 10.5 degrees and 23.5 degrees, such as at least between 10 degrees and 25 degrees, and surgical cutting tool guide 20 is arranged to automatically and non-electrically maintain sight target axis 46 oriented to pass within the offset distance of target point 60 over the entire range of angles α (alpha).

As used in the present application, including in the claims, an angle between two lines is the smaller of the two supplementary angles between the two lines, or equals 90 degrees if the two lines are perpendicular.

Typically, respective sites of primary sight pivot point 50A and secondary sight pivot point 50B are fixed with respect to sight housing 42 (such as by primary and secondary sight pins 82A and 82B, described hereinbelow with reference to FIG. 2). As a result, primary sight pivot point 50A and secondary sight pivot point 50B cannot move with respect to each other or sight housing 42, and primary sight pivot point 50A and secondary sight pivot point 50B are at a fixed distance from each other (second distance D2, described hereinbelow with reference to FIG. 2).

Reference is now made to FIG. 2, which is a schematic illustration of a portion of surgical cutting tool guide 20, in accordance with an application of the present invention. Primary target pivot point 34A, secondary target pivot point 34B, primary sight pivot point 50A, and secondary sight pivot point 50B (a) are not arranged as a parallelogram at any of the angles α (alpha) of the entire range of angles α (alpha) between first and second lines 62A and 62B, and (b) are arranged a quadrilateral 70. The sides of quadrilateral 70 consist of respective segments of (a) a third line 80 defined by primary sight pivot point 50A and secondary sight pivot point 50B, (b) a fourth line 68 defined by primary sight pivot point 50A and primary target pivot point 34A, (c) a fifth line 86 defined by primary target pivot point 34A and secondary target pivot point 34B, and (d) a sixth line 88 defined by secondary target pivot point 34B and secondary sight pivot point 50B. Quadrilateral 70 has a first angle β (beta) at primary sight pivot point 50A and a second angle γ (gamma) at secondary target pivot point 34B. Typically, the first angle β (beta) equals at least 100 degrees, and the second angle γ (gamma) equals at least 25 degrees, and/or a difference between the first angle β (beta) and the second angle γ (gamma) equals at least 75 degrees over the entire range of angles α (alpha) between first and second lines 62A and 62B.

For some applications, a first distance D1 between primary target pivot point 34A and secondary target pivot point 34B is not equal to a second distance D2 between primary sight pivot point 50A and secondary sight pivot point 50B. For some applications, (a) the lesser of the first and the second distances D1 and D2 is less than 90% (e.g., less than 75%, such as less than 66%) of (b) the greater of the first and the second distances D1 and D2. For some applications, the first distance D1 is less than the second distance D2 (as shown in the figures), while for other applications, the first distance D1 is greater than the second distance D2 (configuration not shown).

Alternatively or additionally, for some applications, a third distance D3 between primary sight pivot point 50A and primary target pivot point 34A is not equal to a fourth distance D4 between secondary sight pivot point 50B and secondary target pivot point 34B. For some applications, (a) the lesser of the third and the fourth distances D3 and D4 is less than 98% of (b) the greater of the third and the fourth distances D3 and D4. For some applications, the third distance D3 is less than the fourth distance D4 (as shown in the figures), while for other applications, the third distance D3 is greater than the fourth distance D4 (configuration not shown).

Reference is made to FIGS. 1 and 2. For some applications, third distance D3 (labeled in FIG. 2) equals at least 80%, no more than 120%, and/or between 80% and 120% of target axis distance D_{TA} (labeled in FIG. 1).

For some applications, as shown in the figures, secondary sight arm 32B is farther from first line 62A than primary sight arm 32A is from first line 62A. In other words, primary sight arm 32A is generally between secondary sight arm 32B and first line 62A. For other applications, secondary sight arm 32B is closer to first line 62A than primary sight arm 32A is to first line 62A (configuration not shown).

Reference is still made to FIG. 2. For some applications, third line 80 defined by primary sight pivot point 50A and secondary sight pivot point 50B is parallel to or forms a sight-target-axis angle of less than 5 degrees (e.g., less than 2.5 degrees) with sight target axis 46 over the entire range of angles α (alpha). For some applications, third line 80 defined by primary sight pivot point 50A and secondary sight pivot point 50B is parallel to sight target axis 46 over the entire range of angles α (alpha).

Reference is still made to FIG. 2. In order to maintain the orientation of sight target axis 46 with respect to target point 60, described hereinabove with reference to FIG. 1, surgical cutting tool guide 20 is arranged to automatically and non-electrically adjust an angle σ (sigma) between sight target axis 46 and fourth line 68 (which is defined by primary sight pivot point 50A and primary target pivot point 34A), as measurement angle λ (lambda) changes (measurement angle λ (lambda) is between first line 62A and fourth line 68). For some applications, surgical cutting tool guide 20 is arranged to automatically and non-electrically adjust the angle α (sigma) with spherical motion.

For some applications, a greatest dimension D_{G} of sight 40, measured in a direction parallel to sight target axis 46, is at least 25 mm, no more than 60 mm, and/or between 25 and 60 mm, and/or at least 30%, no more than 72%, and/or between 30% and 72% of the target axis distance D_{TA}. This dimension may enable insertion of surgical cutting tool guide 20 into an oral cavity, such as described hereinbelow with reference to FIGS. 5A-C.

Reference is still made to FIG. 2. Typically:
sight housing 42 is pivotably attached to primary sight arm 32A by a primary sight pin 82A such that sight housing 42 and primary sight arm 32A are rotatable with respect to each other about a primary sight pin axis on which primary sight pivot point 50A is located,
sight housing 42 is pivotably attached to secondary sight arm 32B by a secondary sight pin 82B such that sight housing 42 and secondary sight arm 32B are rotatable with respect to each other about a secondary sight pin axis on which secondary sight pivot point 50B is located; the primary sight pin axis and the secondary sight pin axis are not coaxial,
target arm 30 is pivotably attached to primary sight arm 32A by a primary target pin 84A such that primary sight arm 32A and target arm 30 are rotatable with respect to each other about a primary target pin axis upon which primary target pivot point 34A is located, and/or
target arm 30 is pivotably attached to secondary sight arm 32B by a secondary target pin 84B such that secondary sight arm 32B and target arm 30 are rotatable with respect to each other about a secondary target pin axis upon which secondary target pivot point 34B is located; the primary target pin axis and the secondary sight pin axis are not coaxial.

Primary sight pivot point 50A, secondary sight pivot point 50B, primary target pivot point 34A, and secondary target pivot point 34B define a plane. Typically, the primary sight pin axis, the secondary sight pin axis, the primary target pin axis, and the secondary sight pin axis are perpendicular to the plane. (As used in the present application, including in the claims, if four points are not coplanar, to “define a plane” means to define a best-fit plane derived using the least square orthogonal distances between the points and the best-fit plane.)

Reference is still made to FIG. 2. For some applications, surgical cutting tool guide 20 comprises a plurality of visually-sensible fiducial designators 66 disposed about primary target pivot point 34A, which indicate a measurement angle λ (lambda) between (a) first line 62A and (b) fourth line 68 defined by primary sight pivot point 50A and primary target pivot point 34A. For some applications, visually-sensible fiducial designators 66 are arranged to display the measurement angle λ (lambda) in a range of at least 25 degrees to 45 degrees. Because the size of any given surgical cutting tool guide 20 is known, visually-sensible fiducial designators 66 may alternatively or additionally indicate the distance between sight 40 and target point 60 (e.g., between 30 and 55 mm), which linearly correlates with measurement angle λ (lambda) in the range of at least 25 degrees to 60 degrees.

Reference is now made to FIGS. 3A and 3B, which are schematic illustrations of surgical cutting tool guide 20 with respective different orientations of primary sight arm 32A with respect to target arm 30 (and to respective different orientations of secondary sight arm 32B with respect to target arm 30), in accordance with an application of the present invention. In FIGS. 3A and 3B, by way of example, surgical cutting tool guide 20 is shown with primary sight arm 32A oriented with respect to target arm 30 such that angle α (alpha) is approximately 11 degrees and 22 degrees, respectively.

As can be seen in FIGS. 3A and 3B, surgical cutting tool guide 20 is arranged to automatically and non-electrically maintain sight target axis 46 oriented to pass within the offset distance of a target point 60 over an entire range of angles α (alpha) that extends at least between 11 degrees and 22 degrees. In other words, surgical cutting tool guide 20 is arranged such that sight 40 automatically and non-electrically assumes an orientation that directs surgical cutting tool 22 toward target point 60 (within the offset distance) over the entire range of angles α (alpha), including 11 degrees and 22 degrees, as well as all values therebetween.

As mentioned hereinabove with reference to FIG. 1, typically intersection point 64 at which first and second lines 62A and 62B intersect moves along first line 62A and/or second line 62B over the entire range of angles α (alpha). Intersection point 64 is thus at different locations along first line 62A in FIGS. 3A and 3B.

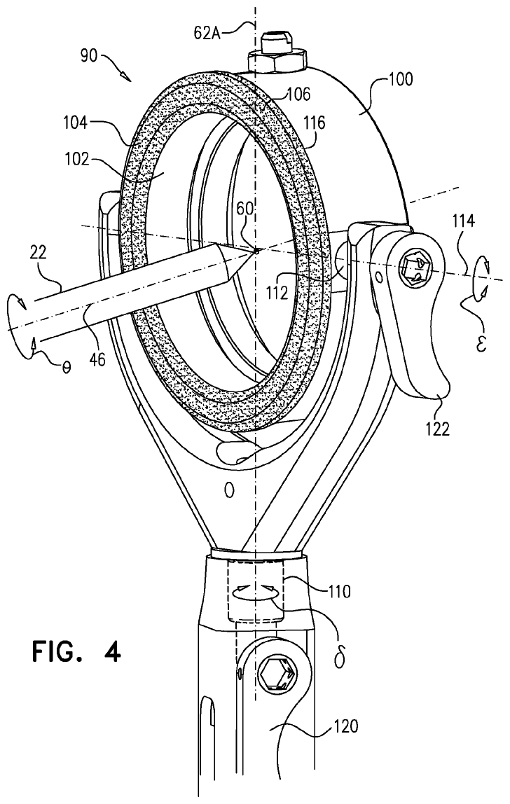

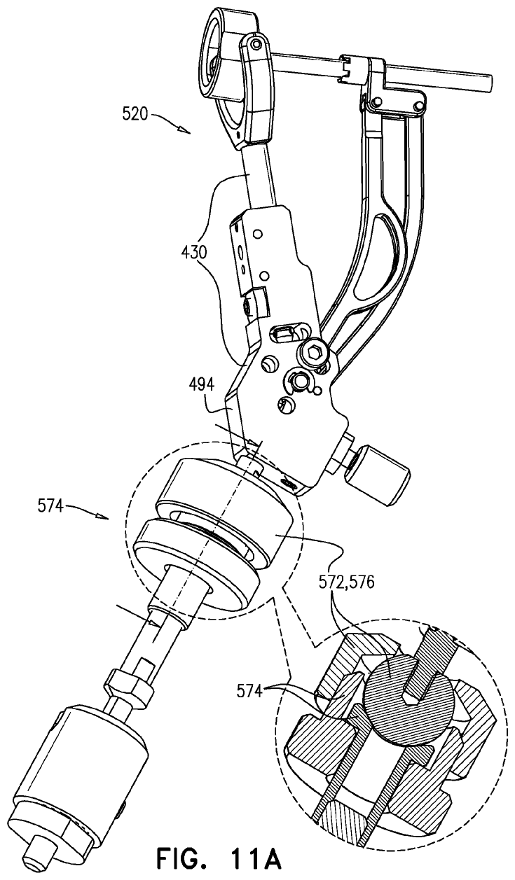

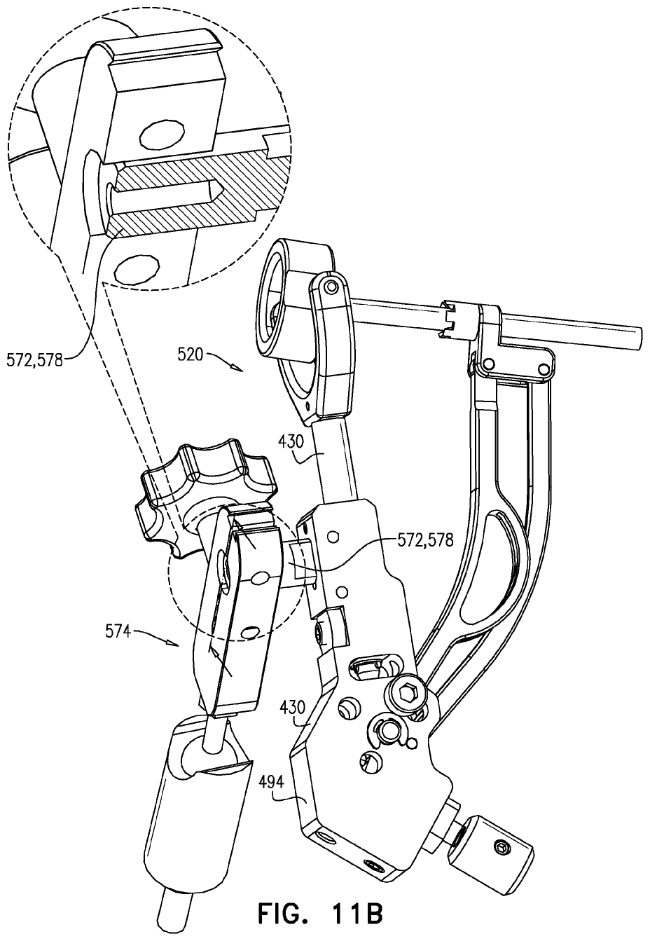

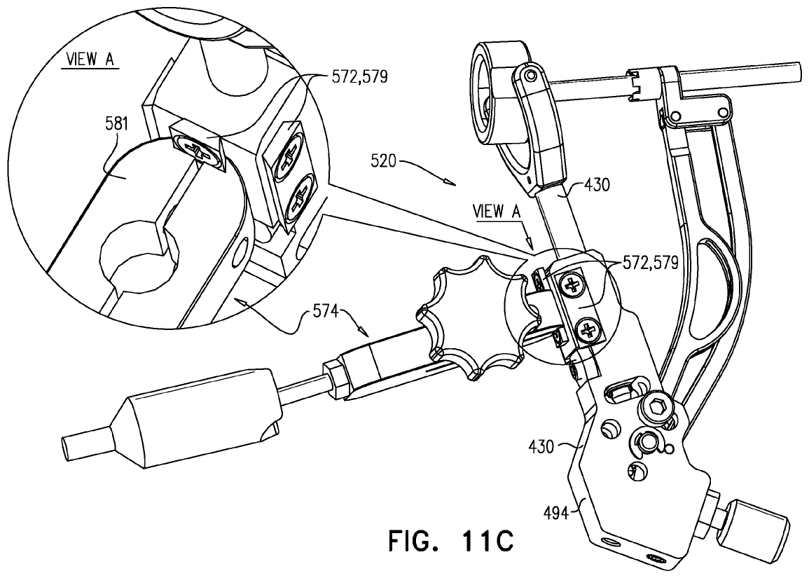

Reference is again made to FIG. 1, and is additionally made to FIG. 4, which is a schematic illustration of a target-stabilization unit 90 of surgical cutting tool guide 20, in accordance with an application of the present invention. Typically, surgical cutting tool guide 20 further comprises target-stabilization unit 90, which is disposed at a distal end 92 of target arm 30, and is configured to stabilize target point 60 with respect to a body of a patient, such as with respect to a zygomatic bone. Optionally, for some applications, target-stabilization unit 90 is stabilized with respect to the zygomatic bone using an external mounting device 574, such as described hereinbelow with reference to FIGS. 11A-C; in these applications, external mounting device 574 stabilizes target-stabilization unit 90 with respect to the zygomatic bone, and target-stabilization unit 90 in turn stabilizes target point 60 with respect to the zygomatic bone. Alternatively, an external mounting device 574 is not used to stabilize target-stabilization unit 90.

Typically, surgical cutting tool guide 20 is arranged to automatically and non-electrically maintain sight target axis 46 oriented to pass through target-stabilization unit 90 over the entire range of angles α (alpha).

For some applications, target-stabilization unit 90 is shaped so as to define a target housing 100 that is disposed around at least 180 degrees (e.g., at least 270 degrees, such as 360 degrees) of target point 60 (and sight target axis 46). Target housing 100 visually indicates the location of target point 60 to the surgeon. For some applications, target housing 100 is shaped so as to define a surface 102 facing target point 60 (and sight target axis 46); for example, surface 102 may be circular or circularly arcuate. Typically, target housing 100 entirely surrounds target point 60 (and sight target axis 46), such as shown in the figures; optionally, surface 102 facing target point 60 (and sight target axis 46) is annular. For example, annular surface 102 may be elliptical, typically circular, such as shown. Typically, target point 60 (and sight target axis 46) is centered in the target housing (i.e., target point 60 (and sight target axis 46) is disposed at a centroid of the space surrounded by annular surface 102). For some applications, target housing 100 is shaped as a ring, and, optionally, target point 60 (and sight target axis 46) is centered in the ring. Optionally, a surface 104 of target housing 100 that faces sight housing 42, and thus is pressed against tissue of the subject, is soft or padded.

Reference is still made to FIGS. 1 and 4. For some applications, surgical cutting tool guide 20 comprises:
a first joint 110 that facilitates relative rotation of target housing 100 about first line 62A over a first entire range of angles δ (delta) that extends at least 90 degrees (e.g., at least 120 degrees); target point 60 moves, with respect to target housing 100, by (a) less than 2% (typically, less than 1%, ideally 0%) of the target axis distance D_{TA} (labeled in FIG. 1) during rotation of target housing 100 over the first entire range of angles δ (delta), and/or (b) less than 2 mm (typically, less than 1 mm, ideally 0 mm);
a second joint 112 that facilitates relative rotation of target housing 100 about a line 114 perpendicular to both first line 62A and sight target axis 46 over a second entire range of angles ϵ (epsilon) that extends at least 90 degrees (e.g., at least 120 degrees); target point 60 moves, with respect to target housing 100, by less than 2% (typically, less than 1%, ideally 0%) of the target axis distance D_{TA} (labeled in FIG. 1) during rotation of target housing 100 over the second entire range of angles ϵ (epsilon), and/or (b) less than 2 mm (typically, less than 1 mm, ideally 0 mm); and/or
a third joint 116 that facilitates relative rotation of an element of target housing 100 about sight target axis 46 over a third entire range of angles θ (theta) that extends at least 90 degrees (e.g., at least 120 degrees, such as 360 degrees); target point 60 moves, with respect to target housing 100, by less than 2% (typically, less than 1%, ideally 0%) of the target axis distance D_{TA} (labeled in FIG. 1) during rotation of target housing 100 over the third entire range of angles θ (theta), and/or (b) less than 2 mm (typically, less than 1 mm, ideally 0 mm).

Thus, in these applications, surgical cutting tool guide 20 provides four degrees of freedom: three degrees of spherical movement around target point 60 (as shown in FIG. 4) plus one degree of movement of target point 60 with respect to sight 40 (as shown in FIGS. 1, 2, and 3A-B). Similarly, for applications in which surgical cutting tool guide 20 comprises only two of the three joints 112, 112, and 116, surgical cutting tool guide 20 provides three degrees of freedom: two degrees of spherical movement around target point 60 (as shown in FIG. 4) plus one degree of movement of target point 60 with respect to sight 40 (as shown in FIGS. 1, 2, and 3A-B).

For some applications, the element of target housing 100 that is rotatable about sight target axis 46 comprises a ring 106, which optionally is soft or padded. For some applications, surgical cutting tool guide 20 comprises first joint 110 and second joint 112 that facilitate relative rotation of target housing 100 about first line 62A over the first entire range of angles δ (delta), and about line 114 over the second entire range of angles ϵ (epsilon), respectively. Target point 60 moves, with respect to target housing 100, by less than 2% (typically, less than 1%, ideally 0%) of the target axis distance D_{TA} (labeled in FIG. 1) during rotation of target housing 100 over the first entire range of angles δ (delta) and the second entire range of angles ϵ (epsilon).

For some applications, surgical cutting tool guide 20 comprises first joint 110, second joint 112, and third joint 116 that facilitate relative rotation of target housing 100 about first line 62A over the first entire range of angles δ (delta), relative rotation of target housing 100 about line 114 over the second entire range of angles ϵ (epsilon), and relative rotation of an element of target housing 100 about sight target axis 46 over the third entire range of angles θ (theta), respectively. Target point 60 moves, with respect to target housing 100, by less than 2% (typically, less than 1%, ideally 0%) of the target axis distance D_{TA} (labeled in FIG. 1) during rotation of target housing 100 over the first entire range of angles δ (delta), the second entire range of angles ϵ (epsilon), and the third entire range of angles θ (theta).

Reference is still made to FIGS. 1 and 4, and is additionally made to FIG. 2. For some applications, surgical cutting tool guide 20 comprises a first lock 120, which is configured, when locked, to rotationally lock first joint 110, thereby preventing relative rotation of target housing 100 about first line 62A; when unlocked, first lock 120 does not prevent such relative rotation. Alternatively or additionally, for some applications, surgical cutting tool guide 20 comprises a second lock 122, which is configured, when locked, to rotationally lock second joint 112, thereby preventing relative rotation of target housing 100 about line 114; when unlocked, second lock 122 does not prevent such relative rotation.

Reference is again made to FIG. 1. For some applications, surgical cutting tool guide 20 comprises a locking mechanism 130, which is configured to lock sight 40 with respect to target-stabilization unit 90, i.e., at a fixed distance from target-stabilization unit 90. For some applications, locking mechanism 130 comprises a ratchet 132, which comprises teeth 134 and a spring-loaded pawl 136 that allows motion of primary and secondary sight arms 32A and 32B toward, but not away from, target arm 30.

Reference is now made to FIGS. 5A-C, which are schematic illustrations of the use of surgical cutting tool guide 20 for drilling a bore into a zygomatic bone 200 for anchoring a zygomatic implant 220 (labeled in FIG. 5C), in accordance with an application of the present invention.

As shown in FIG. 5A, sight 40, as well as portions of primary and secondary sight arms 32A and 32B, are inserted through the mouth into an oral cavity 202. The curvature of primary and secondary sight arms 32A and 32B, if provided, may assist with this insertion. It is noted that the curvature does not affect the fundamental geometry of surgical cutting tool guide 20, which is affected for primary and secondary sight arms 32A and 32B by fourth line 68 and sixth line 88, labeled in FIG. 2.

Target point 60 is positioned at or near a malar point 204 (the most prominent point on zygomatic bone 200). For some applications, target-stabilization unit 90 is placed against skin of a cheek 206, which both positions target point 60 and stabilizes target point 60 with respect to zygomatic bone 200. Target-stabilization unit 90 typically assumes an appropriate orientation by itself, because of the anatomy and the degrees of freedom of motion provided by first joint 110, second joint 112, and/or third joint 116, described hereinabove with reference to FIGS. 1 and 4. After target-stabilization unit 90 has assumed the appropriate orientation, if provided, first lock 120 and/or second lock 122, described hereinabove with reference to FIGS. 1, 2, and 4, are typically locked to maintain the orientation.

Typically, target housing 100 is disposed against the skin partially or entirely around malar point 204.

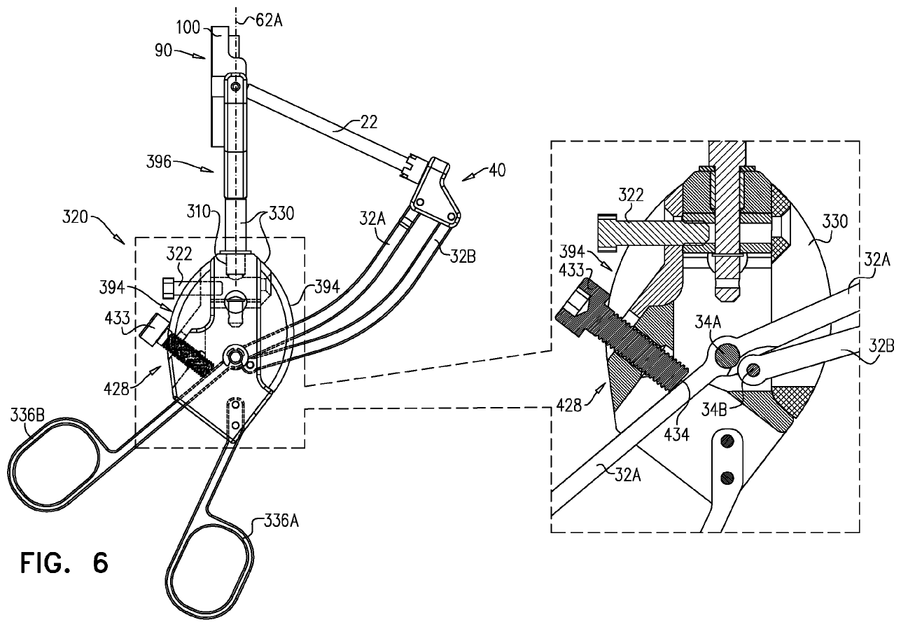

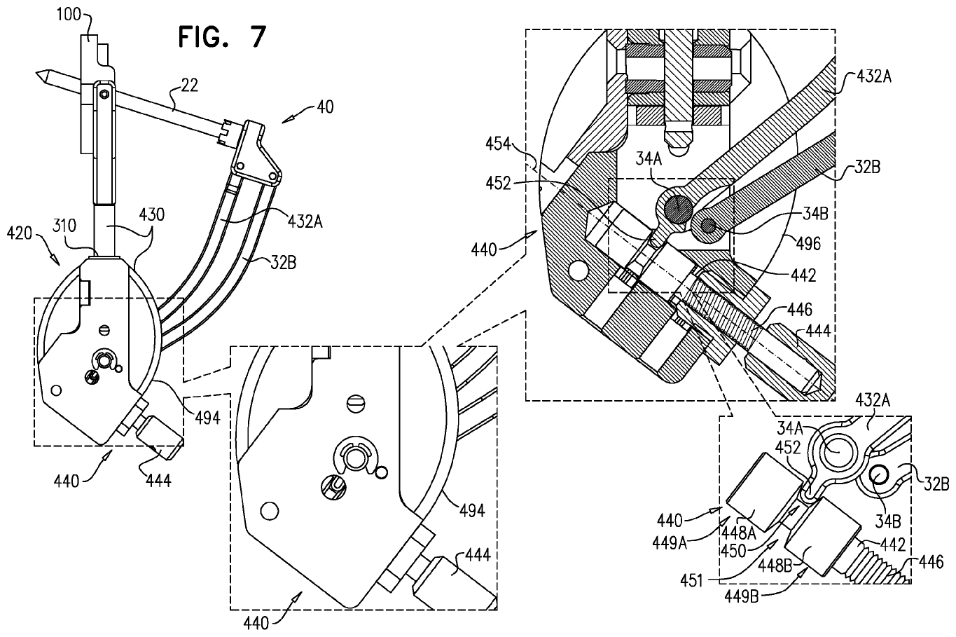

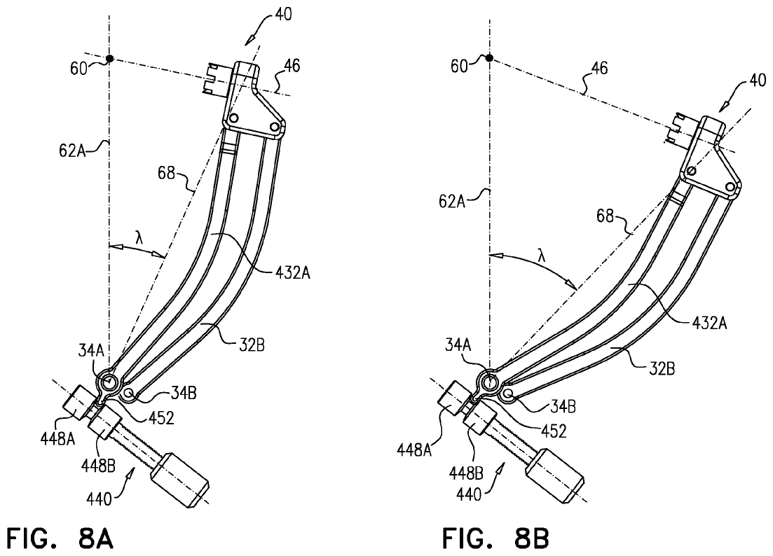

As shown in FIG. 5B, sight 40 is positioned near a desired implantation site 208 of the maxilla (a working surface of the bone), and is pushed tightly against implantation site 208 by bringing sight 40 closer to target-stabilization unit 90, by bringing primary and secondary sight arms 32A and 32B closer to target arm 30. For some applications, primary and secondary sight arms 32A and 32B are brought closer to target arm 30 by bringing finger loops 36A and 36B, which are disposed outside oral cavity 202, closer together, or using locking mechanism 428, described hereinbelow with reference to FIG. 6, or using arm-adjustment mechanism 440, described hereinbelow with reference to FIGS. 7 and 8A-B. Target-stabilization unit 90 and sight 40 are thus arranged to grasp a bony part of the body therebetween. In configurations in which surgical cutting tool guide 20 comprises locking mechanism 130, such as described hereinabove with reference to FIG. 1, the locking mechanism locks sight 40 with respect to target-stabilization unit 90, thereby maintaining sight 40 clamped tightly against implantation site 208 and target-stabilization unit 90 tightly against the skin of cheek 206 at or near malar point 204, and sight 40 at a fixed position and orientation with respect to the anatomy, including zygomatic bone 200. Optionally, in order to release any undesired constrains, sight 40 is gently slightly moved away from the target-stabilization unit and again brought closer to the target-stabilization unit.

As shown in the transition between FIG. 5A and FIG. 5B, as sight 40 is brought closer to target-stabilization unit 90, surgical cutting tool guide 20 automatically and non-electrically maintains the orientation of sight passage 44 toward target point 60, even as the angle α (alpha) changes as the arms are brought closer together.

As a result of these features, the surgeon can use a single hand for positioning and clamping surgical cutting tool guide 20 in the proper position for cutting into zygomatic bone 200. Target point 60 is ex vivo, and surgical cutting tool guide 20 provides non-invasive fixation of target point 60. In addition, the surgeon does not need to calculate or otherwise ascertain the angle α (alpha) before sight 40 is inserted into oral cavity 202.

As shown in FIG. 5C, surgical cutting tool 22, such as a drill bit, is advanced through sight passage 44, and used to drill the bore into zygomatic bone 200. Sight passage 44 directs surgical cutting tool 22 toward target point 60 (within the offset distance), regardless of the particular angle α (alpha), so long as angle α (alpha) is within the range of angles α (alpha) provided by surgical cutting tool guide 20, as described hereinabove with reference to FIGS. 1-3B. Surgical cutting tool guide 20 thus prevents accidental penetration of an orbital cavity 210 by surgical cutting tool 22. To the extent that any deviation may inadvertently occur using surgical cutting tool guide 20, the deviation is lateral, rather than medial, anterior, or superior. Surgical cutting tool guide 20 may also facilitate the performance of a flapless technique (in conventional zygomatic dental implant procedures, a flap is opened to visually check the trajectory of the drill).

Reference is again made to FIG. 1. For some applications in which surgical cutting tool guide 20 is configured for drilling a bore into zygomatic bone 200 for anchoring a zygomatic implant, such as described hereinabove with reference to FIGS. 5A-C, target axis distance D_{TA} is at least 50 mm (e.g., at least 65 mm), and/or no more than 85 mm, such as 78 mm or 83 mm. For some of these applications, when angle α (alpha) equals 11 degrees, a distance between target point 60 and a point on sight 40 closest to target point 60 equals at least 5 mm, no more than 30 mm, and/or between 5 and 30 mm, such as 25 mm, and when angle α (alpha) equals 22 degrees, the distance equals at least 30 mm, no more than 450 mm, and/or between 30 and 450 mm, such as 60 mm. The distance varies as angle α (alpha) varies between 11 and 22 degrees. For some of these applications, the offset distance described hereinabove with reference to FIG. 1 is less than 1 mm, typically less than 0.5 mm. For some of these applications, greatest dimension D_{G} of sight 40, described hereinabove with reference to FIG. 2, is at least 15 mm, no more than 50 mm, and/or between 15 and 50 mm, such as 16 mm. For some of these applications, a length of sight passage 44, measured along sight target axis 46, is at least 10 mm, no more than 60 mm, and/or between 10 and 60 mm, such between 10 and 11 mm.

Thus, for applications in which the target axis distance D_{TA} is at least 50 mm, the less than 2% of the target axis distance D_{TA} mentioned above, with reference to FIGS. 1 and 4 regarding first, second, and third joints 110, 112, and 116, equals less than 1 mm. In addition, for applications in which the target axis distance D_{TA} is at least 50 mm, the less than 5% of the target axis distance D_{TA} mentioned above, with reference to FIG. 1 regarding the offset distance, equals less than 2.5 mm, and the less than 3% of the target axis distance D_{TA} mentioned above, with reference to FIG. 1 regarding the offset distance, equals less than 1.5 mm.

Reference is now made to FIG. 6, which is a schematic illustration of a surgical cutting tool guide 320, in accordance with an application of the present invention. Surgical cutting tool guide 320 is for use with surgical cutting tool 22, such as described herein. Surgical cutting tool 22 is not an element of surgical cutting tool guide 320. Other than as described hereinbelow, surgical cutting tool guide 320 is identical to surgical cutting tool guide 20, described hereinabove with reference to FIGS. 1-5C, and like reference numerals refer to like parts.

Like in surgical cutting tool guide 20, primary sight arm 32A of surgical cutting tool guide 320 is typically, but not necessarily, shaped so as to define a finger loop 336B, and a target arm 330 of surgical cutting tool guide 320 is typically, but not necessarily, shaped so as to define a finger loop 336A. In addition, target arm 330 comprises a central housing 394, which, in configurations in which finger loop 336A is provided, is disposed between finger loop 336A and a distal portion 396 of target arm 330. Central housing 394 of target arm 330 includes primary target pivot point 34A and secondary target pivot point 34B. Other than as described herein, target arm 330 has the features of target arm 30 of surgical cutting tool guide 20, as described hereinabove with reference to FIGS. 1-5C.

Finger loops 336A and 336B facilitate grasping of surgical cutting tool guide 320 by a surgeon, and adjusting the orientation of primary sight arm 32A with respect to target arm 30 (and the orientation of secondary sight arm 32B with respect to target arm 30).

For some applications, surgical cutting tool guide 320 comprises a first joint 310 that facilitates relative rotation of target housing 100 about first line 62A, in the same manner as first joint 110 of surgical cutting tool guide 20, as described hereinabove with reference to FIGS. 1 and 4. For some of these applications, surgical cutting tool guide 320 comprises a first lock 322 fixed to central housing 394. First lock 322 has the same function as first lock 120 of surgical cutting tool guide 20, described hereinabove with reference to FIGS. 1, 2, and 4.

For some applications, central housing 394 of surgical cutting tool guide 320 comprises a locking mechanism 428, which is configured to lock sight 40 with respect to target-stabilization unit 90, i.e., at a locked distance from target-stabilization unit 90. For some applications, locking mechanism 428 comprises a locking screw 433, and central housing 394 is shaped so as to define an internally threaded channel through which locking screw 433 is disposed. A distal end 434 of locking screw 433 is arranged to come in contact with a proximal portion of primary sight arm 32A at a location proximal to primary target pivot point 34A (i.e., primary target pivot point 34A is between the proximal location and the distal end of primary sight arm 32A). Such contact prevents motion of sight 40 away from target-stabilization unit 90. Typically, at the beginning of a procedure, locking screw 433 is unscrewed from central housing 394 sufficiently to not interfere with motion of primary sight arm 32A. The surgeon brings primary and secondary sight arms 32A and 32B closer to target arm 30 by bringing finger loops 336A and 336B closer together, such as described hereinabove with reference to FIG. 5B, mutatis mutandis. The surgeon then tightens locking screw 433 until distal end 434 thereof comes in contact with the proximal portion of primary sight arm 32A, thereby maintaining sight 40 clamped tightly against implantation site 208 and target-stabilization unit 90 tightly against the skin of cheek 206 at or near malar point 204, such as described hereinabove with reference to FIG. 5B, mutatis mutandis.

Reference is now made to FIG. 7, which is a schematic illustration of a surgical cutting tool guide 420, in accordance with an application of the present invention. Surgical cutting tool guide 420 is for use with surgical cutting tool 22, such as described herein. Surgical cutting tool 22 is not an element of surgical cutting tool guide 420. Other than as described hereinbelow, surgical cutting tool guide 420 is identical to surgical cutting tool guide 20, described hereinabove with reference to FIGS. 1-5C, and like reference numerals refer to like parts.

A target arm 430 comprises a central housing 494, which includes primary target pivot point 34A and secondary target pivot point 34B. Other than as described herein, target arm 430 has the features of target arm 30 of surgical cutting tool guide 20, as described hereinabove with reference to FIGS. 1-5C. Unlike in surgical cutting tool guide 20, a primary sight arm 432A of surgical cutting tool guide 320 is typically not shaped so as to define a finger loop, and target arm 330 of surgical cutting tool guide 420 is typically not shaped so as to define a finger loop. For some applications, surgical cutting tool guide 420 comprises first joint 310 and, optionally, first lock 322, such as described hereinabove with reference to FIG. 6 regarding surgical cutting tool guide 320.

For some applications, central housing 494 of surgical cutting tool guide 420 comprises an arm-adjustment mechanism 440, which is configured to adjust the orientation of primary sight arm 432A with respect to target arm 430 (and the orientation of secondary sight arm 32B with respect to target arm 430). For some applications, arm-adjustment mechanism 440 comprises a lead screw 442, which is coupled to central housing 494 such that lead screw 442 can rotate with respect to central housing 494. Lead screw 442 is shaped so as to define a user-control knob 444, which protrudes from, and is accessible from outside of, central housing 494, and a threaded lead screw portion 446. Lead screw 442 and user-control knob 444 may together serve as an arm manipulator. Central housing 494 (e.g., a lead screw nut thereof) is shaped so as to define an internally threaded channel through which threaded lead screw portion 446 is disposed. Rotation of user-control knob 444 causes rotation of threaded lead screw portion 446 of lead screw 442, which in turn causes axial motion of lead screw 442 with respect to central housing 494. The end of user-control knob 444 facing central housing 494 may also serve to prevent over-advancement of lead screw 442 into central housing 494.

Reference is still made to FIG. 7, and is additionally made to FIGS. 8A-B, which are schematic illustrations of two axial positions of lead screw 442 and the resulting relative positions of primary and secondary sight arms 432A and 32B with respect to target point 60, in accordance with an application of the present invention. Lead screw 442 is further shaped so as to define first and second limiting radial protrusions 448A and 448B at different first and second axial regions 449A and 449B, respectively, along lead screw 442. Typically, but not necessarily, first and second limiting radial protrusions 448A and 448B are rotationally symmetrical around a central longitudinal axis 454 of lead screw 442. A proximal end 450 of primary sight arm 432A is shaped so as to define an engaging protrusion 452, which is aligned with an axial region 451 of lead screw 442 that is axially between first and second limiting radial protrusions 448A and 448B. Lead screw 442 and primary sight arm 432A are arranged such that axial motion of lead screw 442 causes one of limiting radial protrusions 448A or 448B to axially engage and push engaging protrusion 452 (which of limiting radial protrusions 448A and 448B axially engages engaging protrusion 452 depends on the direction of axial motion of lead screw 442), resulting in both (a) axial motion of engaging protrusion 452 along central longitudinal axis 454 of lead screw, and (b) rotational motion of engaging protrusion about primary target pivot point 34A. The rotational motion of engaging protrusion 452 in turn causes rotation of primary sight arm 432A about primary target pivot point 34A.

This rotation moves primary sight arm 432A, as well as secondary sight arm 32B, with respect to target arm 430 (and target point 60), as described hereinabove with reference to FIG. 1, mutatis mutandis. The shape of an internal space of central housing 494 in which limiting radial protrusions 448A and 448B are confined, optionally together with the end of user-control knob 444 facing central housing 494, restricts the axial motion of limiting radial protrusions 448A and 448B, and thus of engaging protrusion 452, preventing engaging protrusion 452 from disengaging from axial region 451 that is axially between first and second limiting radial protrusions 448A and 448B. Limiting radial protrusions 448A and/or 448B and/or engaging protrusion 452 may be shaped like cams.

As described hereinabove with reference to FIG. 2, as primary and secondary sight arms 432A and 32B move with respect to target point 60, measurement angle λ (lambda) changes. (Measurement angle λ (lambda) is between first line 62A and fourth line 68, described hereinabove with reference to FIG. 1.)

Reference is still made to FIGS. 7 and 8A-B. Arm-adjustment mechanism 440 also serves as a lock. When user-control knob 444 is not rotated, it remains rotationally fixed because of friction and a geometric lock between threaded lead screw portion 446 of lead screw 442 and the internally threaded channel of central housing 494. As a result, arm-adjustment mechanism 440 locks sight 40 with respect to target-stabilization unit 90, i.e., at a locked distance from target-stabilization unit 90. The surgeon brings primary and secondary sight arms 432A and 32B closer to target arm 430 by rotating user-control knob 444 until sight 40 is clamped tightly against implantation site 208 and target-stabilization unit 90 tightly against the skin of cheek 206 at or near malar point 204, such as described hereinabove with reference to FIG. 5B, mutatis mutandis. The surgeon then ceases to rotate user-control knob 444, and user-control knob 444 maintains the relative dispositions of primary and secondary sight arms 432A and 32B and target arm 430.

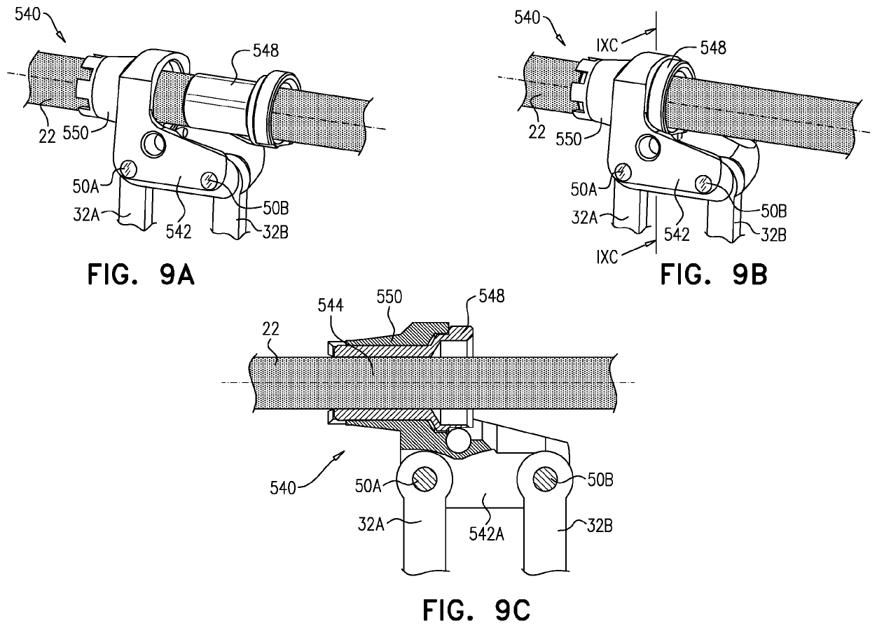

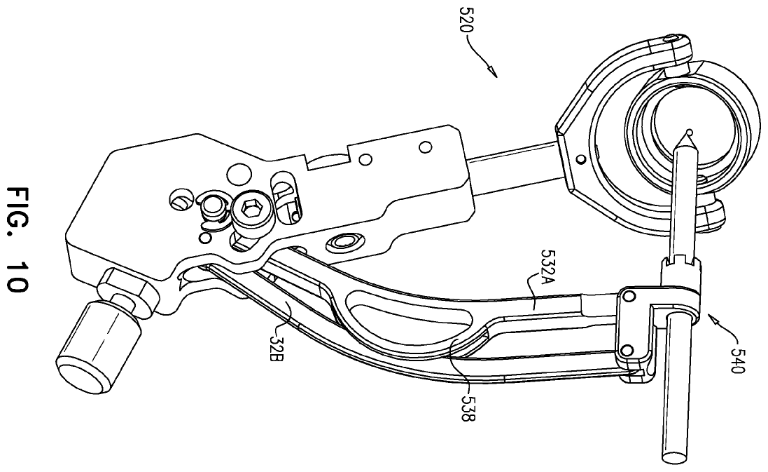

Reference is now made to FIGS. 9A-C, which are schematic illustrations of a sight 540, in accordance with an application of the present invention. Other than as described below, sight 540 is identical to sight 40, described hereinabove with reference to FIGS. 1-5C. Sight 540 comprises a sight housing 542, which is shaped so as to define a fixed external sleeve 550. Sight 540 further comprises an internal guiding sleeve 548, which functions as a sight tube, and is shaped so as to define a sight passage 544 for aligning surgical cutting tool 22 with sight target axis 46, as described regarding sight passage 44 hereinabove with reference to FIG. 1. Internal guiding sleeve 548 is removably insertable into fixed external sleeve 550. Typically, a plurality of internal guiding sleeves 548 are provided, which have different internal diameters, thereby allowing the surgeon to select the diameter of sight passage 544. Typically, external sleeve 550 is rigid, and internal guiding sleeve 548 is rigid. For some applications, external sleeve 550 is configured to be placed in contact with implantation site 208, as described hereinabove regarding sight 40 with reference to FIG. 5B.

Reference is now made to FIG. 10, which is a schematic illustration of a surgical cutting tool guide 520, in accordance with an application of the present invention. Surgical cutting tool guide 520 is for use with surgical cutting tool 22, such as described herein. Surgical cutting tool 22 is not an element of surgical cutting tool guide 520.

Other than as described hereinbelow, surgical cutting tool guide 520 is similar to surgical cutting tool guide 420, described hereinabove with reference to FIGS. 7 and 8A-B, in combination with sight 540, described hereinabove with reference to FIGS. 9A-C, and like reference numerals refer to like parts. In general, surgical cutting tool guide 520 has certain features that reduce gaps, strengthen the guide, and improve its accuracy compared to surgical cutting tool guide 420. For example, a primary sight arm 452A of surgical cutting tool guide 520 comprises an arcuate member 538, which slidingly engages secondary sight arm 32B to stabilize the primary and secondary sight arms with respect to each other. Alternatively, secondary sight arm 32B may comprise the arcuate member.

Reference is now made to FIGS. 11A-C, which are schematic illustrations of respective configurations of surgical cutting tool guide 520, in accordance with respective applications of the present invention. These configurations may also be implemented in combination with surgical cutting tool guide 20, surgical cutting tool guide 320, surgical cutting tool guide 420, or surgical cutting tool guide 620, mutatis mutandis. In these configurations, surgical cutting tool guide 520 (typically, target arm 430, e.g., central housing 494, as shown in FIG. 11A) is shaped so as to define one or more interfaces 572 (e.g., gripping interfaces) configured to be coupled to an interface (e.g., a gripper) of an external mounting device 574. External mounting device 574 is stabilized with respect to the zygomatic bone the patient, typically by fixedly mounting external mounting device 574 to a conventional oral surgery surgical chair or bed in which the patient is positioned, with his or her head stabilized with respect to the chair, or to a mounting support pole near the chair or bed, or to a head fixation unit, all as is known in the art. By stabilizing external mounting device 574 with respect to the zygomatic bone, and using the one or more interfaces 572 to stabilize target arm 430 with respect to external mounting device 574, target point 60 is indirectly stabilized with respect to the zygomatic bone. External mounting device 574 is typically not an element of surgical cutting tool guide 520, though it optionally may be.

For example, the one or more interfaces 572 may comprise:
a mounting ball and socket 576, which defines a coupling interface, such as a female coupling interface (such as shown in FIG. 11A),
a circular mounting rod 578 (such as shown in FIG. 11B); for example, circular mounting rod 578 may have an external diameter of 6, 8, or 10 mm, or any other diameter; typically, the interface of external mounting device 574 includes a slotted hole for placement around rod 578 and a tightening knob/screw, as is known in the art, and/or
one or more male dovetail joints 579 (such as shown in FIG. 11C), i.e., protrusions that are trapezoidal in cross-section, as is known in the art, for mounting with a female dovetail cavity in a gripper interface 581 of external mounting device 574.

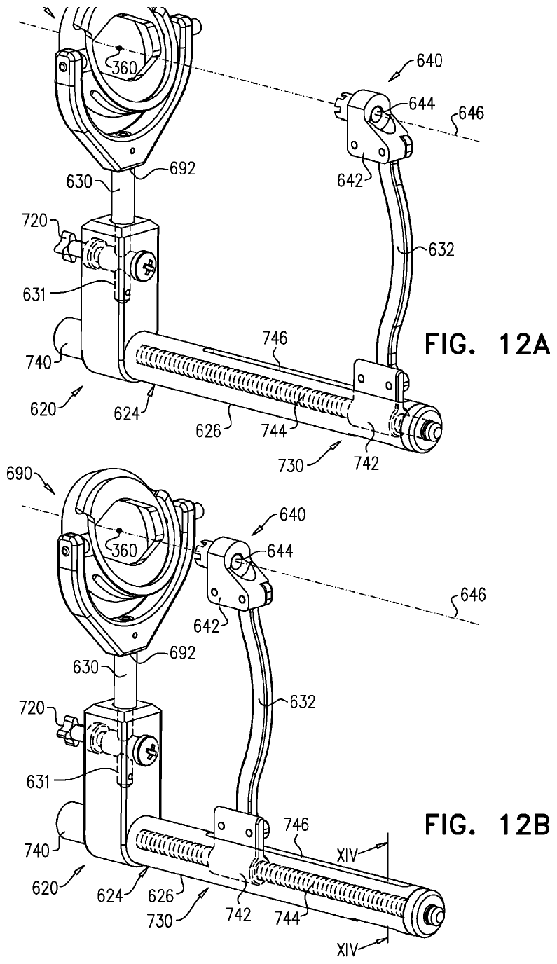

Reference is now made to FIGS. 12A-B, which are schematic illustration of a surgical cutting tool guide 620, in accordance with an application of the present invention. Surgical cutting tool guide 620 is for use with surgical cutting tool 22, such as described herein. Surgical cutting tool 22 is not an element of surgical cutting tool guide 620. Although not shown in FIGS. 12A-B, surgical cutting tool 22 is shown schematically in FIG. 1 and is used in the same way with surgical cutting tool guide 620. For some applications, surgical cutting tool guide 620 is used to drill a bore into a zygomatic bone for anchoring a zygomatic implant, such as described hereinabove with reference to FIGS. 5A-C for surgical cutting tool guide 20, mutatis mutandis.

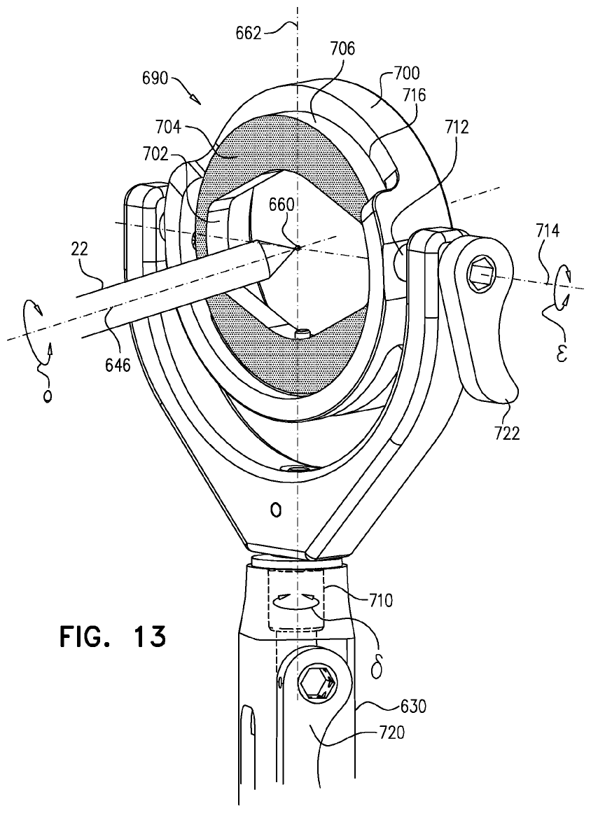

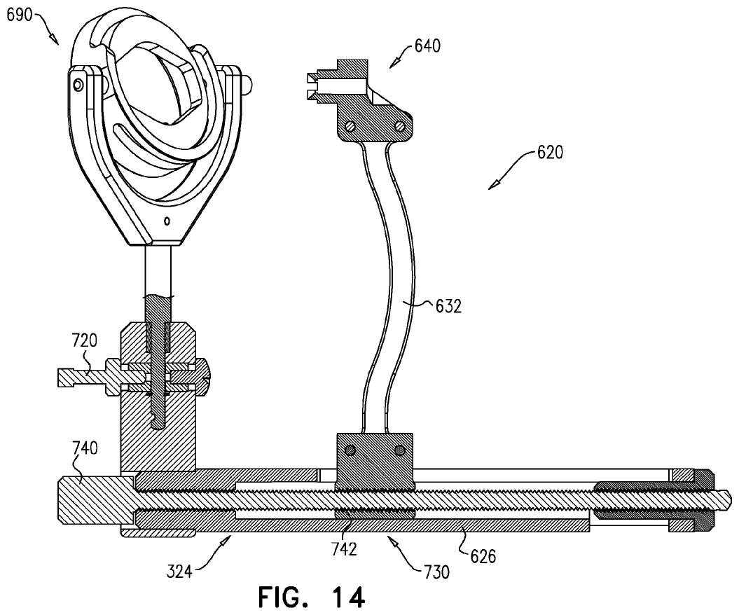

Reference is further made to FIG. 13, which is a schematic illustration of a target-stabilization unit 690 of surgical cutting tool guide 620, in accordance with an application of the present invention. Typically, target-stabilization unit 690 is similar or identical to target-stabilization unit 90, described hereinabove with reference to FIG. 4, and may implement any of the techniques thereof. Similarly, target-stabilization unit 90 may implement any of the techniques of target-stabilization unit 690.

Reference is made to FIGS. 12A-B and 13. Surgical cutting tool guide 620 typically comprises:
a main body 624, which is shaped so as to define an elongate support structure 626;
a target arm 630, a proximal portion 631 of which is attached to main body 624;
a target-stabilization unit 690, which is disposed at a distal end 692 of target arm 630, and is configured to stabilize a target point 660 with respect to the body of the patient;
a sight 640, which comprises a sight housing 642, which defines a sight passage 644 for aligning surgical cutting tool 22 with a sight target axis 646; and
one or more sight arms, typically exactly one sight arm 632, which is attached to sight 640 and slidably attached to elongate support structure 626, such that sight arm 632 can move linearly and assume a plurality of axial positions along elongate support structure 626, thereby permitting relative movement between sight 640 and target arm 630; sight 640 is typically attached to a distal end of sight arm 632.

FIGS. 12A and 12B show sight arm 632 at two different axial positions along elongate support structure 626.

For some applications, sight 640 is generally similar to sight 40 of surgical cutting tool guide 20, described hereinabove with reference to FIGS. 1-5C, and may implement any of the features of sight 40. Alternatively, for some applications, sight 640 is generally similar to sight 540, described hereinabove with reference to FIG. 9, and may implement any of the features of sight 540.

For some applications, all or a portion of sight arm 632 is curved. The curvature, if provided, may assist with the insertion of surgical cutting tool guide 620 into the oral cavity, such as described hereinabove with reference to FIGS. 5A-C for surgical cutting tool guide 20. It is noted that the curvature does not affect the fundamental geometry of surgical cutting tool guide 620.

Surgical cutting tool guide 620 is arranged to automatically and non-electrically maintain sight target axis 646 oriented to pass within an offset distance of target point 660 at all of the axial positions of sight arm 632 along elongate support structure 626. Typically, the offset distance equals less than 2.5 mm, e.g., less than 1.5 mm, such as less than 1 mm or less than 0.5 mm, such that sight 640 directs sight passage 644 toward target point 660 at all of the axial positions of sight arm 632 along elongate support structure 626.

Reference is again made to FIG. 13. For some applications, target-stabilization unit 690 is shaped so as to define a target housing 700 that is disposed around at least 180 degrees (e.g., at least 270 degrees, such as 360 degrees) of target point 660. Target housing 700 visually indicates the location of target point 660 to the surgeon. For some applications, target housing 700 is shaped so as to define a surface 702 facing target point 660 (and sight target axis 646). Typically, target housing 700 entirely surrounds target point 660 (and sight target axis 646), such as shown in the figures; optionally, surface 702 facing target point 660 (and sight target axis 646) is annular. For example, annular surface 702 may be elliptical, typically circular, or polygonal, such as shown. Typically, target point 660 (and sight target axis 646) is centered in the target housing (i.e., target point 660 (and sight target axis 646) is disposed at a centroid of the space surrounded by annular surface 702). For some applications, target housing 700 is shaped as a ring, and, optionally, target point 660 (and sight target axis 646) is centered in the ring. Optionally, a surface 704 of target housing 700 that faces sight housing 642, and thus is pressed against tissue of the subject, is soft or padded. Typically, target housing 700 is generally similar to target housing 100 of surgical cutting tool guide 20, described hereinabove with reference to FIGS. 1-5C, and may implement any of the features of target housing 100.

Reference is still made to FIGS. 12A-B and 13. For some applications, surgical cutting tool guide 620 comprises:
a first joint 710 that facilitates relative rotation of target housing 700 about a target-arm axis 662 of target arm 630 over an entire range of angles δ (delta) that extends at least 90 degrees; target point 660 moves, with respect to target housing 700, by less than 2 mm (typically, less than 1 mm, ideally 0 mm) during rotation of target housing 700 over the entire range of angles δ (delta);
a second joint 712 that facilitates relative rotation of target housing 700 about a line 714 perpendicular to both target-arm axis 662 of target arm 630 and sight target axis 646 over an entire range of angles ϵ (epsilon) that extends at least 90 degrees (e.g., at least 120 degrees); target point 660 moves, with respect to target housing 700, by less than 2 mm (typically, less than 1 mm, ideally 0 mm) during rotation of target housing 700 over the entire range of angles ϵ (epsilon); and/or
a third joint 716 that facilitates relative rotation of an element of target housing 700 about sight target axis 646 over an entire range of angles θ (theta) that extends at least 90 degrees (e.g., at least 120 degrees, such as 360 degrees); target point 660 moves, with respect to target housing 700, by less than 2 mm (typically, less than 1 mm, ideally 0 mm) during rotation of target housing 700 over the entire range of angles θ (theta).

Thus, in these applications, surgical cutting tool guide 620 provides four degrees of freedom: three degrees of spherical movement around target point 660 (as shown in FIG. 13) plus one degree of linear movement of target point 660 with respect to sight 640 (as shown in FIGS. 12A-B). Similarly, for applications in which surgical cutting tool guide 620 comprises only two of the three joints 712, 712, and 716, surgical cutting tool guide 620 provides three degrees of freedom: two degrees of spherical movement around target point 660 (as shown in FIG. 13) plus one degree of linear movement of target point 660 with respect to sight 640 (as shown in FIGS. 12A-B).

For some applications, the element of target housing 700 that is rotatable about sight target axis 646 comprises a ring 706, which optionally is soft or padded. For some applications, surgical cutting tool guide 620 comprises first joint 710 and second joint 712 that facilitate relative rotation of target housing 700 about target-arm axis 662 of target arm 630 over the first entire range of angles δ (delta), and about line 714 over the second entire range of angles ϵ (epsilon), respectively. Target point 660 moves, with respect to target housing 700, by less than 2 mm (typically, less than 1 mm, ideally 0 mm) during rotation of target housing 700 over the first entire range of angles δ (delta) and the second entire range of angles ϵ (epsilon).

For some applications, surgical cutting tool guide 620 comprises first joint 710, second joint 712, and third joint 716 that facilitate relative rotation of target housing 700 about target-arm axis 662 of target arm 630 over the first entire range of angles δ (delta), relative rotation of target housing 700 about line 714 over the second entire range of angles ϵ (epsilon), and relative rotation of an element of target housing 700 about sight target axis 646 over the third entire range of angles θ (theta), respectively. Target point 660 moves, with respect to target housing 700, by less than 2 mm (typically, less than 1 mm, ideally 0 mm) during rotation of target housing 700 over the first entire range of angles δ (delta), the second entire range of angles ϵ (epsilon), and the third entire range of angles θ (theta).

Reference is again made to FIGS. 12A-B and 13. For some applications, surgical cutting tool guide 620 comprises a first lock 720, which is configured, when locked, to rotationally lock first joint 710, thereby preventing relative rotation of target housing 700 about target-arm axis 662; when unlocked, first lock 720 does not prevent such relative rotation. Alternatively or additionally, for some applications, surgical cutting tool guide 620 comprises a second lock 722, which is configured, when locked, to rotationally lock second joint 712, thereby preventing relative rotation of target housing 700 about line 714; when unlocked, second lock 722 does not prevent such relative rotation.

Reference is now made to FIG. 14, which is a schematic cross-sectional view of surgical cutting tool guide 620, in accordance with an application of the present invention. For some applications, target-stabilization unit 690 and sight 640 are arranged to grasp a bony part of the body therebetween. For some applications, surgical cutting tool guide 620 is configured to allow sight arm 632 to move linearly along elongate support structure 626 a total distance of at least 30 mm, no more than 80 mm (e.g., no more than 40 mm), and/or between 30 and 80 mm, e.g., between 30 and 40 mm. For some applications, main body 624 comprises a mechanical control element 740, e.g., comprising a knob, which is disposed remotely from sight arm 632, and is configured to mechanically and non-electrically linearly move sight arm 632 along elongate support structure 626.

For some applications, sight arm 632 includes (i.e., is either shaped so as to define or comprises) a nut 742 that is shaped so as to define an internal threaded channel. Main body 624 further comprises an elongate lead screw 744, which is disposed within an elongate channel defined within elongate support structure 626 such that elongate lead screw 744 is rotatable within the channel and axially fixed with respect to elongate support structure 626. A thread of elongate lead screw 744 is threadingly engaged with the internal threaded channel of nut 742, such that rotation of elongate lead screw 744 causes axial movement of nut 742, and thus of sight arm 632. Mechanical control element 740 is attached to or integral with one end of elongate lead screw 744, to allow the user to rotate the lead screw. (Mechanical control element 740 is shown in FIG. 14 disposed at one end of elongate lead screw 744; mechanical control element 740 may alternatively be disposed at the opposite end (configuration not shown).) Elongate support structure 626 typically is shaped so as to define an axially-oriented elongate slot 746 along at least an axial portion of elongate support structure 626, and a portion of sight arm 632 passes through elongate slot 746 between inside to outside elongate support structure 626. The portion of sight arm 632 is axially slidable in slot 746 along elongate support structure 626. Slot 746 is configured to inhibit (e.g., prevent) rotational movement of sight arm 632 about a central longitudinal axis of elongate support structure 626.

For some applications, surgical cutting tool guide 620 further comprises a locking mechanism 730, which is configured to lock sight 640 with respect to target-stabilization unit 690, typically by friction and a geometric lock between lead screw 744 and the internally threaded channel of nut 742.

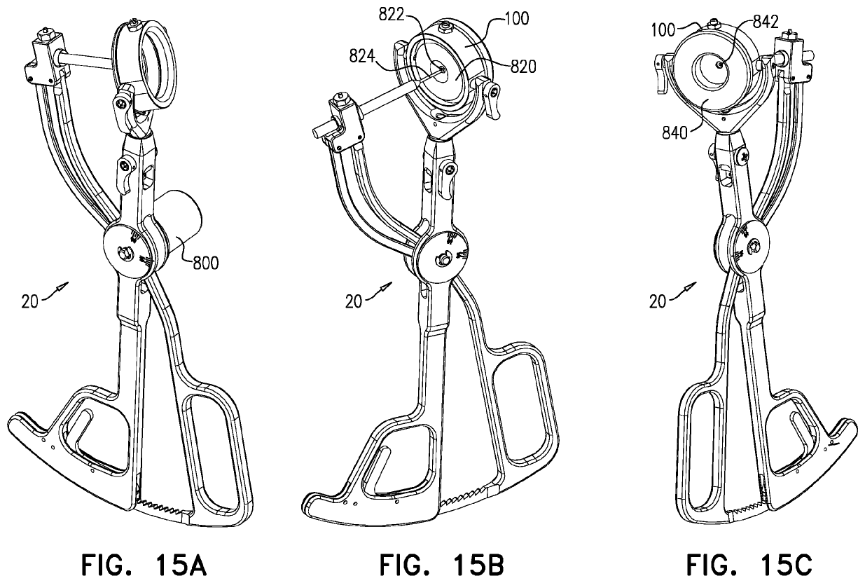

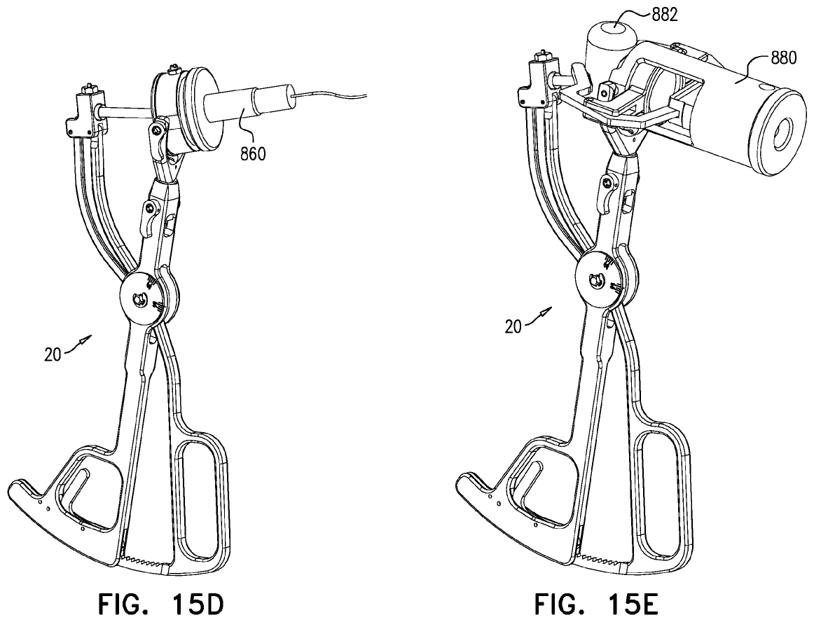

Reference is now made to FIGS. 15A-E, which are schematic illustrations of optional components that may be used in conjunction with surgical cutting tool guide 20 or surgical cutting tool guide 620, in accordance with respective applications of the present invention. The component described with reference to FIG. 15A is for use in conjunction with surgical cutting tool guide 20, surgical cutting tool guide 320, or surgical cutting tool guide 420. The components described with reference to FIGS. 15B-E are for use in conjunction with surgical cutting tool guide 20, surgical cutting tool guide 320, surgical cutting tool guide 420, or surgical cutting tool guide 620, although for the sake of brevity are only shown in use with surgical cutting tool guide 20.

In the configuration shown in FIG. 15A, surgical cutting tool guide 20 further comprises an electrical unit 800 that is coupled to surgical cutting tool guide 20 at primary target pivot point 34A, and is configured to perform direct measurement of measurement angle λ (lambda). For example, electrical unit 800 may comprise a rheostat or a rotary potentiometer. The measured angle is optionally transmitted to a computer, a mobile device, and/or microcontroller, and may be processed in order to generate output parameters such as measurement angle λ (lambda), a distance, and a recommended implant.

In the configuration shown in FIG. 15B, surgical cutting tool guide 20, surgical cutting tool guide 320, surgical cutting tool guide 420, or surgical cutting tool guide 620 further comprises a removable calibration disc 820, which defines a hole 822 at its center. Calibration disc 820 is used prior to use of the surgical cutting tool guide 20 in a surgical procedure, to check whether the surgical cutting tool guide 20 is properly calibrated and undamaged. Calibration disc 820 is placed within the opening of target housing 100 or target housing 700. An elongate test tool 824 is passed through sight passage 44 or sight passage 644. A user checks whether a distal tip of elongate test tool 824 is able to pass through hole 822 or within a desired offset of hole 822 over an entire range of desired angles of operation of surgical cutting tool guide 20, surgical cutting tool guide 320, or surgical cutting tool guide 420, or over an entire range of axial positions of sight arm 632 of surgical cutting tool guide 620 along elongate support structure 626 of surgical cutting tool guide 620.

In the configuration shown in FIG. 15C, surgical cutting tool guide 20, surgical cutting tool guide 320, surgical cutting tool guide 420, or surgical cutting tool guide 620 further comprises a removable malar centering disc 840, which defines an aiming hole 842 at its center, and is placed within the opening of target housing 100 or target housing 700. After locating malar point 204, as described hereinabove with reference to FIG. 5A, but before placing target-stabilization unit 90 or target-stabilization unit 690 against the skin, the surgeon makes a small mark (e.g., a dot) on the skin at malar point 204. The surgeon places aiming hole 842 over the mark by looking through the aiming hole. The aiming hole may provide better aiming than just using target housing 100 or target housing 700 alone, because the ring of target housing 100 or target housing 700 is substantially larger than the aiming hole. Surface 104 of target housing 100 or surface 704 of target housing 700 is held in place against the skin, and malar centering disc 840 is decoupled from target housing 100 or target housing 700, such as by unscrewing, and removed from the side of target housing 100 or 700 opposite surface 104 or 704. For example, aiming hole 842 may have a diameter of 5 mm.

In the configuration shown in FIG. 15D, surgical cutting tool guide 20, surgical cutting tool guide 320, surgical cutting tool guide 420, or surgical cutting tool guide 620 further comprises a removable proximity sensor 860, which is placed within the opening of target housing 100 or target housing 700. Proximity sensor 860 is configured to sense when a distal tip of a drill bit is at a specified distance from target point 60 or target point 660, and generate a signal. For example, use of proximity sensor 860 may prevent the drill tip from puncturing the skin. For some applications, proximity sensor 860 comprises an inductive sensor, which is configured to sense the metal of the tip of a convention drill bit. Proximity sensor 860 may be coupled to housing 100 or housing 700 after the housing has been placed surrounding malar point 204.

In the configuration shown in FIG. 15E, surgical cutting tool guide 20, surgical cutting tool guide 320, surgical cutting tool guide 420, or surgical cutting tool guide 620 further comprises a bone gripper 880, which is coupled to target housing 100 or target housing 700 and extends beyond target housing 100 or 700 toward sight 40 or sight 640. Bone gripper 880 is configured to grip a bone 882 (e.g., a surgically-exposed bone), e.g., a femoral bone, and may be used for example, in a non-dental procedure, such as an orthopedic procedure. Typically, bone gripper 880 is rotatable about sight target axis 46 or 646, and thus may serve as the element of target housing 100 or 700 that is rotatable about sight target axis 46 or 646 instead of ring 106, as described hereinabove with reference to FIGS. 1 and 4, or ring 706, as described hereinabove with reference to FIGS. 15A-B and 13. Optionally, a lock is provided for rotationally locking bone gripper 880 with respect to sight target axis 46 or 646. Although surgical cutting tool guide 20, surgical cutting tool guide 320, surgical cutting tool guide 420, and surgical cutting tool guide 620 have been described herein as for use for dental applications, the tool guides may also be used for other applications, such as orthopedic applications.

Reference is made to FIGS. 1-14. For some applications, a method is provided for drilling a bore into zygomatic bone 200 for anchoring zygomatic implant 220. The method comprises:
inserting sight 40, 540, or 640 through a mouth into oral cavity 202;
before, after, or while inserting sight 40, 540, or 640 through a mouth into oral cavity 202, outside oral cavity 202 positioning target point 60 or 660 at or near malar point 204 (by way of example, and not limitation, as shown in FIG. 5A);
positioning the sight near desired implantation site 208 of the maxilla (by way of example, and not limitation, as shown in FIGS. 5A-B); the sight comprises sight housing 42, 542, or 642 that defines sight passage 44 or 644 for aligning surgical cutting tool 22 (e.g., with sight target axis 46 or 646);
orienting sight passage 44 or 644 to point toward target point 60 or 660 (e.g., by orienting sight target axis 46 or 646 to pass within an offset distance (such as 2.5 mm, e.g., 1.5 mm) of target point 60 or 660) (by way of example, and not limitation, as shown in FIGS. 5A-B);
advancing surgical cutting tool 22 through sight passage 44 or 644, toward target point 60 or 660 (by way of example, and not limitation, as shown in FIG. 5C); and
drilling the bore using surgical cutting tool 22 (by way of example, and not limitation, as shown in FIG. 5C).

The method may further implement any of the techniques described hereinabove. These techniques include, but are not limited to the techniques described in the following paragraphs.

For some applications, positioning target point 60 or 660 comprises stabilizing the target point with respect to zygomatic bone 200. For some applications, the target point is stabilized with respect to the zygomatic bone by placing a target-stabilization unit against skin of a cheek. The target-stabilization unit may comprise one of the target-stabilization units described hereinabove. Alternatively, the target-stabilization unit may comprise any element configured to be coupled to the skin (optionally, but not necessarily, with a mark thereon indicative of a location of target point 60 or 660), such as a sticker configured to adhere to the skin; or any fiducial marker. Alternatively, a mark indicating target point 60 or 660 may be made on the skin, such as using ink. For some applications, sight 40, 540, or 640 is positioned near desired implantation site 208 by pushing the sight tightly against desired implantation site 208 by bringing the sight closer to the target-stabilization unit.

For some applications, the target-stabilization unit is shaped so as to define a target housing that is disposed around at least 180 degrees of the target point, and the target-stabilization unit is placed against the skin of the cheek by disposing the target housing against the skin partially or entirely around the malar point.

For some applications, positioning sight 40, 540, or 640 near desired implantation site 208 comprises pushing the sight 40, 540, or 640 tightly against desired implantation site 208. Alternatively or additionally, for some applications, the target-stabilization unit is placed against the skin of the cheek by bringing sight 40, 540, or 640 and the target-stabilization unit closer to each other.

For some applications, sight passage 44 or 644 is oriented to point toward target point 60 or 660 using a surgical cutting tool guide 20, 320, 420, 520, and 620 that is arranged to automatically and non-electrically orient sight passage 44 or 644 to point toward the target point, the surgical cutting tool guide comprising the sight 40, 540, or 640. Alternatively, a surgical cutting tool guide is used that electrically orients the sight passage (configuration not shown).

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising a surgical cutting tool guide for use with a surgical cutting tool, the surgical cutting tool guide comprising:
a target arm;
a primary sight arm, which is pivotably attached to the target arm such that the primary sight arm and the target arm are rotatable with respect to each other about a primary target pivot point, so as to permit relative movement between the primary sight arm and the target arm;
a secondary sight arm, which is pivotably attached to the target arm such that the secondary sight arm and the target arm are rotatable with respect to each other about a secondary target pivot point that does not coincide with the primary target pivot point, so as to permit relative movement between the primary sight arm and the target arm; and
a sight, which comprises a sight housing, which (a) defines a sight passage for aligning the surgical cutting tool with a sight target axis, and (b) is attached to:
the primary sight arm such that the sight housing and the primary sight arm are rotatable with respect to each other about a primary sight pivot point, and
the secondary sight arm such that the sight housing and the secondary sight arm are rotatable with respect to each other about a secondary sight pivot point that does not coincide with the primary sight pivot point,
wherein the surgical cutting tool guide is arranged to automatically and non-electrically maintain the sight target axis oriented to pass within an offset distance of a target point over an entire range of angles that extends at least between 11 degrees and 22 degrees, wherein the angles correspond to respective different orientations of the primary sight arm with respect to the target arm and are measured between (a) a first line that is defined by the target point and the primary target pivot point and (b) a second line that (i) intersects the first line and (ii) intersects and is perpendicular to the sight target axis, and
wherein the offset distance equals less than 5% of a target axis distance measured between the target point and the primary target pivot point, such that the sight directs the sight passage toward the target point over the entire range of angles.

2. The apparatus according to claim 1, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.

3. The apparatus according to claim 1, wherein the offset distance equals less than 3% of the target axis distance.

4. The apparatus according to claim 3, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.

5. The apparatus according to claim 3, wherein the offset distance equals less than 1% of the target axis distance.

6. The apparatus according to claim 5, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.

7. The apparatus according to claim 5, wherein the offset distance equals less than 0.75% of the target axis distance.

8. The apparatus according to claim 7, wherein the entire range of angles that extends at least between 10 degrees and 25 degrees, and wherein the surgical cutting tool guide is arranged to automatically and non-electrically maintain the sight target axis oriented to pass within the offset distance of the target point over the entire range of angles that extends at least between 10 degrees and 25 degrees.

9. The apparatus according to claim 1, wherein the cutting tool is a drill bit, wherein the surgical cutting tool guide is a surgical drill guide, and wherein the sight passage is arranged to guide the surgical drill bit along the sight target axis.

10. The apparatus according to claim 1, wherein the cutting tool is a milling bit, wherein the surgical cutting tool guide is a surgical milling guide, and wherein the sight passage is arranged to guide the surgical milling bit along the sight target axis.

11. The apparatus according to claim 1, wherein the cutting tool is a laser cutting tool, wherein the surgical cutting tool guide is a surgical laser cutting tool guide, and wherein the sight passage is arranged to align the laser cutting tool along the sight target axis.

12. The apparatus according to claim 1, wherein the cutting tool is an ultrasonic cutting tool, wherein the surgical cutting tool guide is a surgical ultrasonic cutting tool guide, and wherein the sight passage is arranged to align the ultrasonic cutting tool with the sight target axis.

13. The apparatus according to claim 1, wherein a third distance between the primary sight pivot point and the primary target pivot point is not equal to a fourth distance between the secondary sight pivot point and the secondary target pivot point.

14. The apparatus according to claim 13, wherein (a) the lesser of the third and the fourth distances is less than 98% of (b) the greater of the third and the fourth distances.

15. The apparatus according to claim 13, wherein the third distance is less than the fourth distance.

16. The apparatus according to claim 1, wherein the secondary sight arm is farther from the first line than the primary sight arm is from the first line.

17. The apparatus according to claim 1, wherein the secondary sight arm runs at least partially alongside the primary sight arm.

18. The apparatus according to claim 1, wherein the target arm and the primary sight arm are shaped so as to define respective finger loops.

19. The apparatus according to claim 1, wherein the surgical cutting tool guide comprises a plurality of visually-sensible fiducial designators disposed about the primary target pivot point, which indicate a measurement angle between (a) the first line and (b) a fourth line defined by the primary sight pivot point and the primary target pivot point.

20. The apparatus according to claim 19, wherein the visually-sensible fiducial designators are arranged to display the measurement angle in a range of at least 25 degrees to 45 degrees.

21. The apparatus according to any one of claims 1-20, wherein the primary target pivot point, the secondary target pivot point, the primary sight pivot point, and the secondary sight pivot point (a) are not arranged as a parallelogram at any of the angles of the entire range of angles between the first and the second lines, and (b) are arranged a quadrilateral.

22. The apparatus according to claim 21,
wherein the quadrilateral has a first angle at the primary sight pivot point and a second angle at the secondary target pivot point, and
wherein a difference between the first and the second angles equals at least 75 degrees over the entire range of angles between the first and the second lines.

23. The apparatus according to any one of claims 1-20, wherein the target arm comprises a central housing, which includes the primary target pivot point and the secondary target pivot point.

24. The apparatus according to claim 23,
wherein the central housing comprises an arm-adjustment mechanism, which comprises a lead screw, which (a) is coupled to the central housing such that the lead screw can rotate with respect to the central housing, and (b) is shaped so as to define:
a user-control knob,
a threaded lead screw portion, wherein the central housing is shaped so as to define an internally threaded channel through which the threaded lead screw portion is disposed,
first and second limiting radial protrusions at different first and second axial regions, respectively, along the lead screw,
wherein a proximal end of the primary sight arm is shaped so as to define an engaging protrusion, which is aligned with an axial region of the lead screw that is axially between the first and the second limiting radial protrusions, and
wherein the lead screw and the primary sight arm are arranged such that axial motion of the lead screw causes axial motion of the engaging protrusion, which in turn causes rotation of the primary sight arm about the primary target pivot point, wherein the rotation moves the primary sight arm with respect to the target arm.

25. The apparatus according to any one of claims 1-20, wherein the surgical cutting tool guide further comprises a target-stabilization unit, which is disposed at a distal end of the target arm, and is configured to stabilize the target point with respect to a body of a patient.

26. The apparatus according to claim 25, wherein the surgical cutting tool guide is arranged to automatically and non-electrically maintain the sight target axis oriented to pass through the target-stabilization unit over the entire range of angles.

27. The apparatus according to claim 25, wherein the target-stabilization unit is shaped so as to define a target housing that is disposed around at least 180 degrees of the target point.

28. The apparatus according to claim 27, wherein the target housing is shaped so as to define a surface facing the target point.

29. The apparatus according to claim 27, wherein the target housing is disposed around at least 270 degrees of the target point.

30. The apparatus according to claim 29, wherein the target housing entirely surrounds the target point.

31. The apparatus according to claim 30, wherein the target housing is shaped so as to define an annular surface facing the target point.

32. The apparatus according to claim 31, wherein the annular surface is circular or polygonal.

33. The apparatus according to claim 30, wherein the target point is centered in the target housing.

34. The apparatus according to claim 27, wherein the surgical cutting tool guide comprises a joint that facilitates relative rotation of the target housing about the first line over an entire range of angles that extends at least 90 degrees, and wherein the target point moves, with respect to the target housing, by less than 2% of the target axis distance during rotation of the target housing over the entire range of angles.

35. The apparatus according to claim 27, wherein the surgical cutting tool guide comprises a joint that facilitates relative rotation of the target housing about a line perpendicular to both the first line and the sight target axis over an entire range of angles that extends at least 90 degrees, and wherein the target point moves, with respect to the target housing, by less than 2% of the target axis distance during rotation of the target housing over the entire range of angles.

36. The apparatus according to claim 27, wherein the surgical cutting tool guide comprises a joint that facilitates relative rotation of an element of the target housing about the sight target axis over an entire range of angles that extends at least 90 degrees, and wherein the target point moves, with respect to the target housing, by less than 2% of the target axis distance during rotation of the target housing over the entire range of angles.

37. The apparatus according to claim 27,
wherein the surgical cutting tool guide comprises joints that facilitate relative rotation of the target housing about:
the first line over a first entire range of angles that extends at least 90 degrees, and
a line perpendicular to both the first line and the sight target axis over a second entire range of angles that extends at least 90 degrees, and
wherein the target point moves, with respect to the target housing, by less than 2% of the target axis distance during rotation of the target housing over the first entire range of angles and the second entire range of angles.

38. The apparatus according to claim 27,
wherein the surgical cutting tool guide comprises joints that facilitate relative rotation of:
the target housing about the first line over a first entire range of angles that extends at least 90 degrees,
the target housing about a line perpendicular to both the first line and the sight target axis over a second entire range of angles that extends at least 90 degrees,
an element of the target housing about the sight target axis over a third entire range of angles that extends at least 90 degrees, and
wherein the target point moves, with respect to the target housing, by less than 2% of the target axis distance during rotation of the target housing over the first entire range of angles, the second entire range of angles, and the third entire range of angles.

39. The apparatus according to claim 25, wherein the target-stabilization unit and the sight are arranged to grasp a bony part of the body therebetween.

40. The apparatus according to claim 39, wherein the surgical cutting tool guide further comprises a locking mechanism, which is configured to lock the sight with respect to the target-stabilization unit.

41. The apparatus according to claim 40,
wherein the target arm comprises a central housing, which includes the primary target pivot point and the secondary target pivot point,
wherein the locking mechanism comprises a locking screw having a distal end arranged to come in contact with a proximal portion of the primary sight arm at a location proximal to the primary target pivot point, and
wherein such contact prevents motion of the sight away from the target-stabilization unit.

42. The apparatus according to any one of claims 1-20, wherein a first distance between the primary target pivot point and the secondary target pivot point is not equal to a second distance between the primary sight pivot point and the secondary sight pivot point.

43. The apparatus according to claim 42, wherein (a) the lesser of the first and the second distances is less than 90% of (b) the greater of the first and the second distances.

44. The apparatus according to claim 43, wherein (a) the lesser of the first and the second distances is less than 75% of (b) the greater of the first and the second distances.

45. The apparatus according to claim 44, wherein (a) the lesser of the first and the second distances is less than 66% of (b) the greater of the first and the second distances.

46. The apparatus according to claim 42, wherein the first distance is less than the second distance.

47. The apparatus according to any one of claims 1-20, wherein a third line defined by the primary sight pivot point and the secondary sight pivot point is parallel to or forms a sight-target-axis angle of less than 5 degrees with the sight target axis over the entire range of angles.

48. The apparatus according to claim 47, wherein the sight-target-axis angle is less than 2.5 degrees over the entire range of angles.

49. The apparatus according to claim 47, wherein the third line defined by the primary sight pivot point and the secondary sight pivot point is parallel to the sight target axis over the entire range of angles.

50. The apparatus according to any one of claims 1-20, for use with an external mounting device having an interface, wherein the surgical cutting tool guide is shaped so as to define one or more interfaces configured to be coupled to the interface of the external mounting device.
